(19) United States
(12) Patent Application Publication
(10) Pub. No.: US 2019/0298387 A1
(43) Pub. Date: Oct. 3, 2019

(54) ANEURYSM TREATMENT COILS

(71) Applicant: Covidien LP, Mansfield, MA (US)

(72) Inventors: Yunguo Qin, Shanghai (CN); Zheng Li, Austin, TX (US); Feilong Jin, Shanghai (CN); Jing Tang, Arlington, MA (US); Xin Wen, Shanghai (CN); Zhimin Fang, Shanghai (CN); Dongliang Song, Shanghai (CN); Shichang Wen, Neijiang City (CN); Yuewen Zheng, Shanghai (CN); Junmin Guo, Shanghai (CN); Quan Zhang, Shanghai (CN); Yuqiang Wang, Shanghai (CN)

(21) Appl. No.: 16/301,223

(22) PCT Filed: May 13, 2016
(86) PCT No.: PCT/CN2016/082046

§ 371 Date: Nov. 13, 2018

(51) Int. Cl. A61B 17/12

(52) U.S. Cl. CPC A61B 17/12145; A61B 2017/00526; A61B 17/12113

(57) ABSTRACT

An implant for treatment of a vascular space can form open loops and closed loops in separate cycles to provide balanced stiffness and flexibility. Such an implant provides both stability for retention within the vascular space and conformability for mitigating forces on the vasculature. Such a configuration can also provide proper coverage over an opening to a vascular space into which the implant is delivered, for reducing or eliminating flow into or out of the body cavity and promoting occlusion of the vascular space.

### CROSS-REFERENCE TO RELATED APPLICATIONS

The present application is a U.S. national phase application of International Application No. PCT/CN2016/082046, filed on May 13, 2016, the entirety of which is incorporated by reference herein.

### TECHNICAL FIELD

The subject technology relates to formation and delivery of implantable devices.

### BACKGROUND

Implants are delivered to a vascular site, such as an aneurysm, of a patient via a microcatheter to occlude or embolize the vascular site. Typically, the implant is engaged at the distal end of either the delivery microcatheter or the guidewire contained within the microcatheter and controllably released therefrom into the vascular site to be treated. The clinician delivering the implant must navigate the microcatheter or guide catheter through the vasculature and, in the case of intracranial aneurysms, navigation of the microcatheter is through tortuous microvasculature. This delivery can be visualized by fluoroscopy or another suitable means. Once the distal tip of the catheter or guidewire is placed in the desired vascular site, the clinician must then begin to articulate the implant in the vascular site to ensure that the implant will be positioned in a manner to sufficiently embolize the site. Once the implant is appropriately positioned, the clinician must then detach the implant from the catheter or guidewire without distorting the positioning of the implant. Detachment can occur through a variety of means, including, electrolytic detachment, chemical detachment, mechanical detachment, hydraulic detachment, and thermal detachment.

Previously, there had been provided 3-dimensional coils which are formed from a straight wire by detachment from the catheter or guidewire. The 3-dimensional coil is typically formed from a metal which upon detachment (e.g., in vivo) reconfigures from the straight wire into a coil shape or confirmation having a secondary structure (i.e., an extended or helically coil confirmation) which under ideal circumstances will comport to the shape of the vascular site to be embolized.

### SUMMARY

One or more embodiments of the subject technology are directed to an implant comprising a 3-dimensional coil designed to optimize packing into a vascular site, such as an aneurysm. It is contemplated that the implant of the invention, due to its secondary shape, is able to substantially conform to a vascular site thereby providing a more effective embolization. The subject technology is illustrated, for example, according to various aspects described below.

According to one or more embodiments of the present disclosure, an embolic implant can include a strand forming, in a relaxed state, (i) first consecutive loops in a first cycle along a first length of the strand and (ii) second consecutive loops in a second cycle along a second length of the strand. Each of the first loops can be an open loop that extends only partially about a corresponding first axis, and each of the second loops can be a closed loop that extends completely about a corresponding second axis that extends through a space within a radially adjacent one of the first loops.

The strand can form, in the relaxed state, a three-dimensional polyhedral shape. In the relaxed state, each of the first loops can contact and share tangent lines with at least one other first loop. A total number of the first loops can be equal to a total number of the second loops. The second consecutive loops can form a radially outermost section of the implant.

The strand can further form, in the relaxed state, consecutive third loops in a third cycle along a third length of the strand, each of the third loops extending about a corresponding third axis that that extends through a space within a radially adjacent one of the second loops. Each of the third loops can be an open loop that extends only partially about the third axis. Each of the third loops can be a closed loop that extends completely about the third axis. The first consecutive loops can form a radially innermost section of the implant.

According to one or more embodiments of the present disclosure, a method of forming an embolic implant can include winding a first length of a strand in a first cycle only partially about each of a plurality of posts extending from a core of a mandrel to form consecutive open first loops, and winding a second length of the strand in a second cycle completely about each of the posts to form consecutive closed second loops.

The first length and the second length can be wound such that the strand forms a three-dimensional polyhedral shape. The first length can be wound such that each of the first loops contacts and shares tangent lines with other first loops. The second length can be wound to form a total number of the second loops equal to a total number of the first loops.

The method can further include winding a third length of the strand in a third cycle about each of the posts to form consecutive third loops. The third length can be wound such that each of the third loops can be an open loop that extends only partially about a corresponding one of the posts. The third length can be wound such that each of the third loops can be a closed loop that extends completely about a corresponding one of the posts.

According to one or more embodiments of the present disclosure, a method of delivering an embolic implant can include providing an implant within a delivery device to a target location while the implant can be in a first, substantially straight configuration, and positioning the implant at the target location such that the implant can be in a secondary configuration in which the implant includes: a strand forming (i) first consecutive loops in a first cycle along a first length of the strand and (ii) second consecutive loops in a second cycle along a second length of the strand, wherein each of the first loops can be an open loop that extends only partially about a corresponding first axis, and each of the second loops can be a closed loop that extends completely about a corresponding second axis that that extends through a space within a radially adjacent one of the first loops.

The implant can be positioned to form, in the secondary configuration, a three-dimensional polyhedral shape. The implant can be positioned, in the secondary configuration, such that each of the first loops contacts and shares tangent lines with other first loops. The implant can be positioned, in the secondary configuration, such that a total number of the first loops can be equal to a total number of the second loops.

The implant can be positioned, in the secondary configuration, to form consecutive third loops in a third cycle along a third length of the strand, each of the third loops extends about a corresponding third axis that that extends through a space within a radially adjacent one of the second loops. The implant can be positioned, in the secondary configuration, such that each of the third loops can be an open loop that extends only partially about the third axis. The implant can be positioned, in the secondary configuration, such that each of the third loops can be a closed loop that extends completely about the third axis.

The implant can be positioned, in the secondary configuration, such that the first consecutive loops form a radially innermost section of the implant. The implant can be positioned, in the secondary configuration, such that the second consecutive loops form a radially outermost section of the implant. The implant can be positioned within an aneurysm.

According to one or more embodiments of the present disclosure, an embolic implant can include a strand forming, in a relaxed state, (i) first consecutive loops in a first cycle along a first length of the strand and (ii) second consecutive loops in a second cycle along a second length of the strand, wherein each of the first loops extends about a corresponding first axis and has a first loop shape, wherein each of the second loops extends about a corresponding second axis that that extends through a space within a radially adjacent one of the first loops and has a second loop shape, different from the first loop shape.

Each of the first loops can be an open loop that extends only partially about the first axis, and each of the second loops can be a closed loop that extends completely about the second axis. A minimum cross-sectional dimension of the first loops can be greater than a minimum cross-sectional dimension of the second loops. A maximum cross-sectional dimension of the first loops can be greater than a maximum dimension of the second loops. Each of the first loops can form a polygon. Each of the second loops can form a circle or an arc of an incomplete circle.

According to one or more embodiments of the present disclosure, a mandrel for forming an embolic implant can include a core and a plurality of posts extending from a surface of the core, each of the posts extending along an axis and having (i) a base section with a first on-face shape and (ii) an extension section with a second on-face shape, different from the first on-face shape.

The base section can be between the core and the extension section. A minimum cross-sectional dimension of the base section can be greater than a minimum cross-sectional dimension of the extension section. A maximum cross-sectional dimension of the base section can be greater than a maximum cross-sectional dimension of the extension section. The base section can form a polygon in cross-section. The extension section can form a circle in cross-section.

According to one or more embodiments of the present disclosure, an embolic implant can include a strand forming, in a relaxed state, first loops and second loops in one or more cycles along a length of the strand; wherein the first loops lie in separate planes on opposite sides of a center of the implant, wherein the first loops both extend about a shared first axis; wherein each of the second loops extends about one of a plurality of second axes that can be not parallel with any other of the second axes of any other of the second loops.

Each of the second axes can intersect with the first axis to form a first angle and a second angle, different from the first angle. Each of the second loops can be closer to one of the first loops than it can be to another of the first loops. Some of the second loops can be closer to a first one of the first loops than to a second one of the first loops and others of the second loops can be closer to the second one of the first loops than to the first one of the first loops.

According to one or more embodiments of the present disclosure, a mandrel for forming an embolic implant can include a core; first posts extending from a surface of the core on opposite sides of the core and along a shared first axis; and second posts extending from the surface of the core along corresponding second axes, wherein each of the second axes can be not parallel with a second axis of any other second post.

Each of the second axes can intersect with the first axis to form a first angle and a second angle, different from the first angle. Each of the second posts can be closer to one of the first posts than another of the first posts. Some of the second posts can be closer to a first one of the first posts than to a second one of the first posts and others of the second posts can be closer to the second one of the first posts than to the first one of the first posts.

According to one or more embodiments of the present disclosure, an embolic implant can include a strand forming, in a relaxed state, first loops along a first section of the strand and second loops along a second section of the strand; wherein each of the first loops extends about one of a plurality of first axes; wherein each of the second loops extends about one of a plurality of second axes; wherein the first axes intersect at a first region, and the second axes intersect at a second region, distinct from the first region; wherein a total number of first loops can be different from a total number of second loops.

The first region can be located between at least two first loops, and the second region can be located between at least two second loops. The first region can be located outside a space bounded by second loops, and the second region can be located outside a space bounded by first loops. The maximum dimension of the first section can be different from a maximum dimension of the second section. The maximum dimension of the first loops can be different from a maximum dimension of the second loops. The first axes can be orthogonal to an axis extending through the first region and the second region, and the second axes can be transverse to the axis. Some of the first loops form, in the relaxed state, a first three-dimensional polyhedral shape, and others of the first loops form, in the relaxed state, a second three-dimensional polyhedral shape, different than the first three-dimensional polyhedral shape.

The strand can further form third loops in a third section, wherein each of the third loops and extends about a third axis, wherein the third axes intersect at a third region, distinct from the first region and the second region.

According to one or more embodiments of the present disclosure, a mandrel for forming an embolic implant can include a first core; first posts extending from a surface of the first core along corresponding first axes, the first axes intersecting at a first region within the first core; a second core; second posts extending from a surface of the second core along corresponding second axes, the second axes intersecting at a second region within the second core and distinct from the first region; wherein a total number of first posts can be different from a total number of second posts.

The first region can be located outside the second core, and the second region can be located outside the first core. The maximum cross-sectional dimension of the first core can be different from a maximum cross-sectional dimension of the second core. The maximum cross-sectional dimension of the first posts can be different from a maximum cross-sectional dimension of the second posts. The first axes can be orthogonal to an axis extending through the first region and the second region, and the second axes can be transverse to the axis.

The mandrel can further include a third core; and third posts extending from a surface of the third core along corresponding third axes, the third axes intersecting at a third region distinct from each of the first region and the second region.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1A shows a plan view of a positioning system and an implant, in accordance with one or more embodiments of the present disclosure.

FIG. 1B shows a closer view of a portion of FIG. 1A.

FIG. 1C shows a plan view of the position system of FIG. 1A within the human body.

FIG. 1D shows a closer view of a portion of FIG. 1C showing the positioning system in partial cross-section and an exemplary implant within the human body, in accordance with one or more embodiments of the present disclosure.

FIG. 1E shows a closer view of a portion of FIG. 1C showing the positioning system in partial cross-section and an exemplary implant within the human body, in accordance with one or more embodiments of the present disclosure.

FIG. 2A shows a partial cross-sectional view of an exemplary positioning system, in accordance with one or more embodiments of the present disclosure.

FIG. 2B shows a side view of another exemplary positioning system, in accordance with one or more embodiments of the present disclosure.

FIG. 3 shows a partial cutaway view of an implant formed in a primary shape as a wound filament strand, in accordance with one or more embodiments of the present disclosure.

FIGS. 4A and 4B show plan views of an implant having a secondary shape, in accordance with one or more embodiments of the present disclosure.

FIG. 5 shows plan views of separate cycles of an implant and a view of the implant including both cycles while in a secondary shape, in accordance with one or more embodiments of the present disclosure.

FIG. 6 shows plan views of separate cycles of an implant and a view of the implant including both cycles while in a secondary shape, in accordance with one or more embodiments of the present disclosure.

FIG. 7A shows a perspective view of a mandrel for imparting a secondary shape to an implant, in accordance with one or more embodiments of the present disclosure.

FIG. 7B shows a perspective view of an implant formed by the mandrel of FIG. 7A, in accordance with one or more embodiments of the present disclosure.

FIG. 8A shows a perspective view of a mandrel for imparting a secondary shape to an implant, in accordance with one or more embodiments of the present disclosure.

FIG. 8B shows a perspective view of an implant formed by the mandrel of FIG. 8A, in accordance with one or more embodiments of the present disclosure.

FIG. 9A shows a perspective view of a mandrel for imparting a secondary shape to an implant, in accordance with one or more embodiments of the present disclosure.

FIG. 9B shows a perspective view of an implant formed by the mandrel of FIG. 9A, in accordance with one or more embodiments of the present disclosure.

FIGS. 10 and 11 show representations of winding patterns for the mandrel of FIG. 8A, in accordance with one or more embodiments of the present disclosure.

FIG. 12A shows a perspective view of a mandrel for imparting a secondary shape to an implant, in accordance with one or more embodiments of the present disclosure.

FIG. 12B shows a perspective view of a mandrel with an implant formed thereon, in accordance with one or more embodiments of the present disclosure.

FIGS. 12C and 12D show perspective views of an implant formed by the mandrel of FIG. 12A, in accordance with one or more embodiments of the present disclosure.

FIG. 13A shows a perspective view of a mandrel for imparting a secondary shape to an implant, in accordance with one or more embodiments of the present disclosure.

FIG. 13B shows a perspective view of a mandrel with an implant formed thereon, in accordance with one or more embodiments of the present disclosure.

FIG. 13C shows a perspective view of an implant formed by the mandrel of FIG. 13A, in accordance with one or more embodiments of the present disclosure.

FIG. 14A shows a perspective view of a mandrel for imparting a secondary shape to an implant, in accordance with one or more embodiments of the present disclosure.

FIG. 14B shows a perspective view of a mandrel with an implant formed thereon, in accordance with one or more embodiments of the present disclosure.

FIGS. 14C and 14D show perspective views of an implant formed by the mandrel of FIG. 14A, in accordance with one or more embodiments of the present disclosure.

FIG. 15A shows a perspective view of a mandrel for imparting a secondary shape to an implant, in accordance with one or more embodiments of the present disclosure.

FIG. 15B shows a perspective view of a mandrel with an implant formed thereon, in accordance with one or more embodiments of the present disclosure.

FIG. 15C shows a perspective view of an implant formed by the mandrel of FIG. 15A, in accordance with one or more embodiments of the present disclosure.

FIG. 15D shows a view of an implant delivered to a target location within a body vessel, in accordance with one or more embodiments of the present disclosure.

FIG. 16A shows a perspective view of a mandrel for imparting a secondary shape to an implant, in accordance with one or more embodiments of the present disclosure.

FIG. 16B shows a perspective view of a mandrel with an implant formed thereon, in accordance with one or more embodiments of the present disclosure.

FIG. 16C shows a perspective view of an implant formed by the mandrel of FIG. 16A, in accordance with one or more embodiments of the present disclosure.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the description.

According to one or more embodiments, the systems and devices disclosed herein can be used in human or veterinary medicine and, more particularly, for the endovascular treatment of intracranial aneurysms and acquired or innate arteriovenous blood vessel malformations and/or fistulas and/or for the embolization of tumors by thromboembolization. For this purpose, components of the various systems and devices disclosed herein can be designed as a coil implant, a filter, a stent, and the like, but may as well possess any other superimposed configuration as may be expedient. In one or more embodiments, the systems and devices disclosed herein may provide various designs and configurations for an aneurysm coil, as especially appropriate for the occlusion of intracranial aneurysms.

According to one or more embodiments, the systems and devices disclosed herein provide enhanced stability of an implant during and after deployment. Such stability promotes retention of the implant within a target site without migration therefrom or movement therein after delivery. The systems and devices disclosed herein further provide an implant with significant flexibility with respect to the target site to conform to the body anatomy without exerting excessive forces thereon. For example, an implant can provide balanced stiffness and flexibility to improve both stability and conformability. Such a configuration can also provide proper coverage over an opening to a body cavity (e.g., aneurysm) into which the implant is delivered, for reducing or eliminating flow into or out of the body cavity and promoting occlusion of the body cavity. Such implants can further be provided as framing coils for supporting yet other implants delivered to an interior space encompassed by the framing coil for enhanced filling and embolization of the target site.

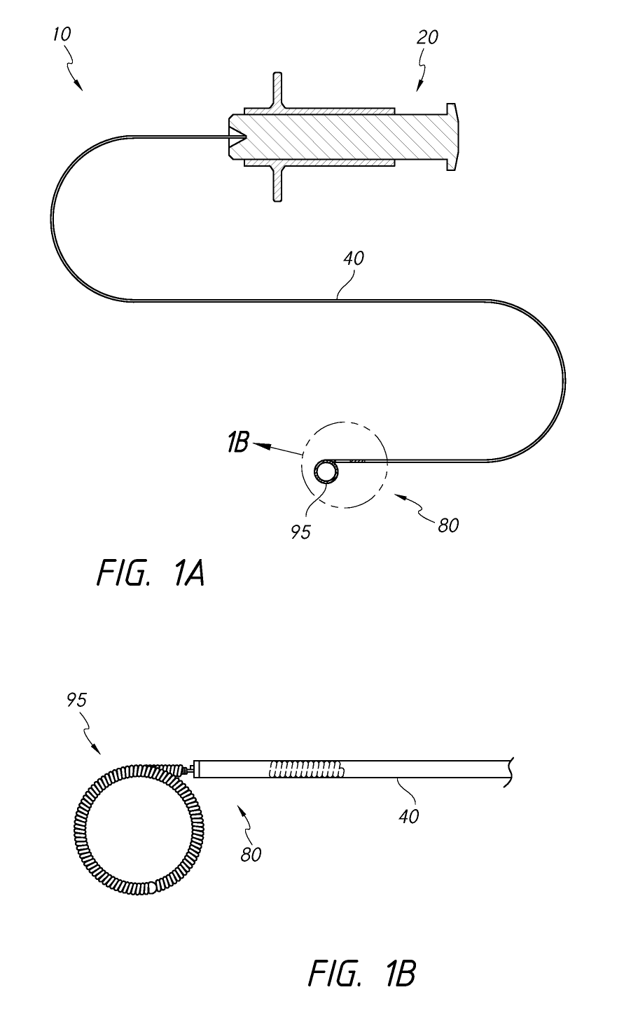

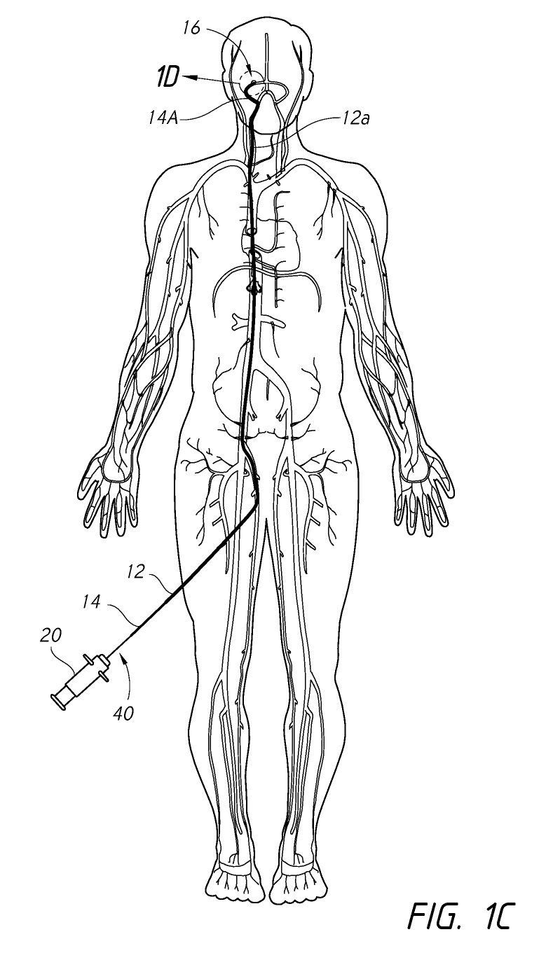

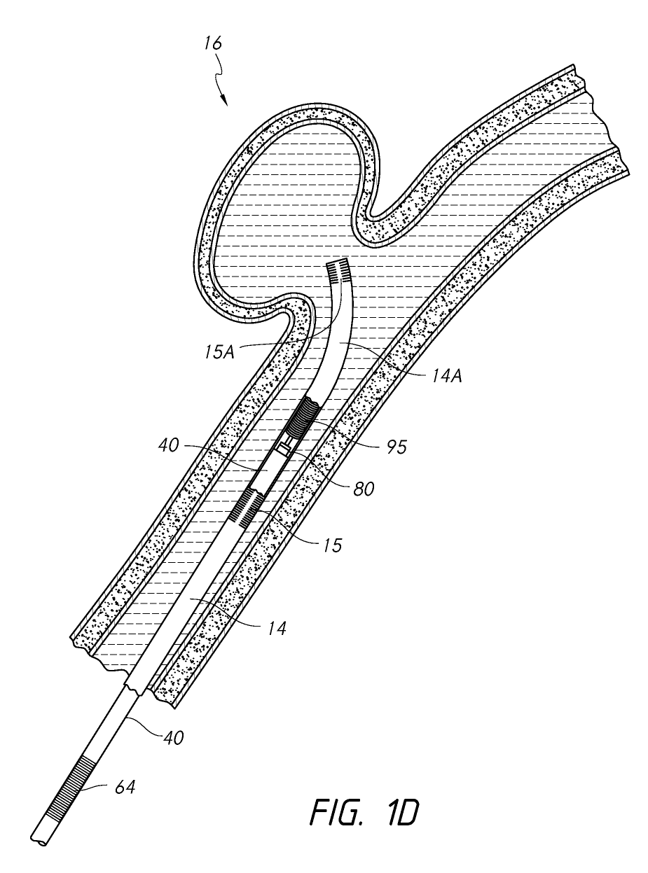

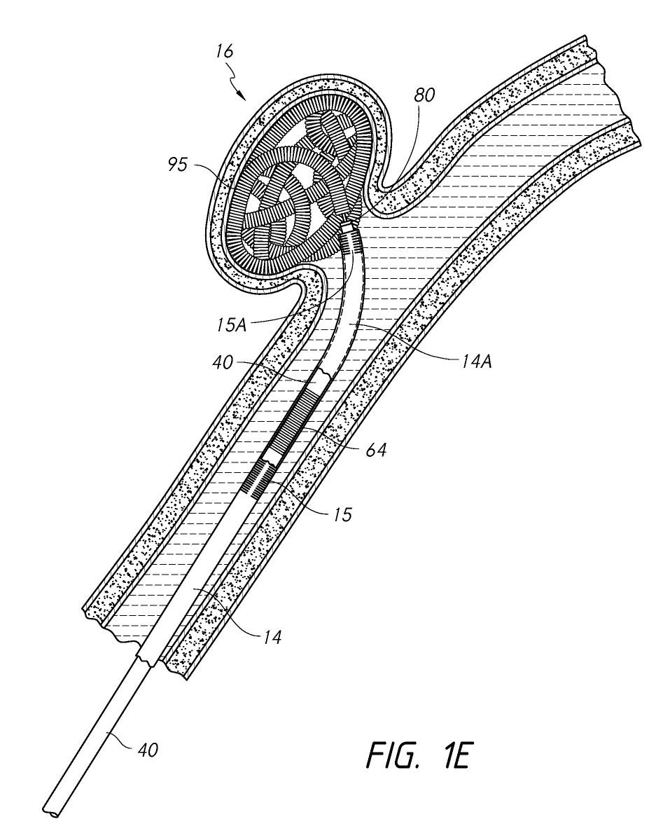

According to one or more embodiments, a vascular implant device can be part of or included with a positioning system 10 such as the one shown in FIGS. 1A-B. The positioning system 10 shown in FIGS. 1A-B includes an actuator 20, a positioner 40 coupled with the actuator 20, and an implant interface 80 at the distal end of the positioner 40. A portion of the implant interface 80 can engage a complementary portion of an implant 95 in order to control the delivery (i.e., securing and detaching) of the implant 95 at the desired location. While the implant 95 is shown or described in several embodiments as comprising an embolic coil, any implant or device that is compatible with the subject technology can be used in lieu of or in conjunction with the coil in accordance with the embodiments described herein.

FIG. 1C shows the positioning system 10 of FIGS. 1A-B used inside a patient's vasculature. In the embodiment shown in FIG. 1C, an operator uses a guide tube or guide catheter 12 to position a delivery tube or microcatheter 14 in a patient's vasculature. This procedure involves inserting the guide catheter 12 into the patient's vasculature through an access point such as the groin, and directing the distal end 12A of the guide catheter 12 through the vascular system until it reaches the carotid artery. After removing a guide wire (not shown) from the guide catheter 12, a microcatheter 14 can be inserted into the guide catheter 12 and the distal end 14a of the microcatheter 14 subsequently exits the guide catheter distal end 12A and can be positioned near the target site 16, such as an aneurysm in the patient's brain.

In the embodiments illustrated in FIGS. 1D and 1E, the microcatheter 14 can include microcatheter markers 15 and 15a that facilitate imaging of the distal end 14a of the microcatheter 14 with common imaging systems. After the distal end 14a reaches the target site 16, the positioning system 10 of the illustrated embodiment is then inserted into the microcatheter 14 to position the implant interface 80 at the distal end of the positioner 40 near the target site 16, as illustrated in FIG. 1D. The implant 95 can be attached to the implant interface 80 prior to inserting the positioning system 10 into the microcatheter 14. This mode of implant delivery is illustrated in FIGS. 1C-E. The delivery of the implant 95 is facilitated by disposing the microcatheter marker 15a near the target site 16, and aligning the microcatheter marker 15 with a positioner marker 64 in the positioner 40 which, when the two markers (markers 15 and 64) are aligned with each other as illustrated in FIG. 1E, indicates to the operator that the implant interface 80 is in the proper position for the release of the implant 95 from the positioning system 10.

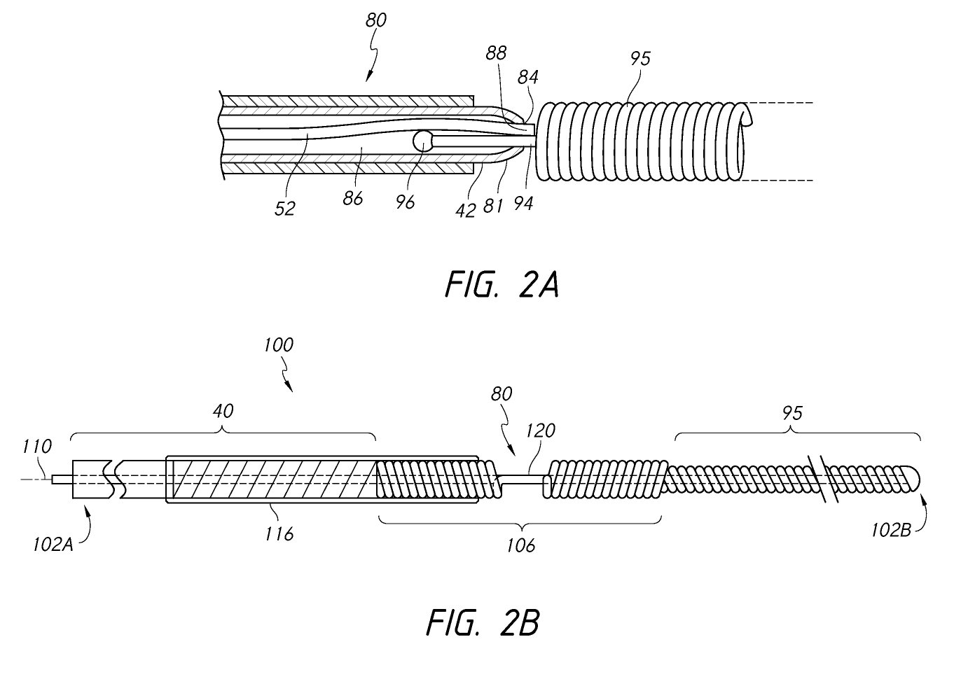

Referring to FIGS. 2A-B, the implant interface 80 is a portion of the positioning system 10 that allows the operator to control the engagement and disengagement of the implant 95 to the positioner 40. As will be appreciated by those skilled in the art, several methods or processes of detaching the implant 95 from the positioner 40 at the implant interface 80 are possible.

Referring specifically to the exemplary embodiment shown in FIG. 2A, a cord 52 can be disposed at the implant interface 80, according to one or more embodiments of the present disclosure. A distal tip 88 of the cord 52 is positioned in the port 84 of the end cap 81 so that it partially obstructs the port 84 when the cord 52 is at its most distally advanced position in the positioner tube 42. The positioner tube 42 and the end cap 81 cooperatively define a cavity 86 within the implant interface 80. The distal tip 88 of the cord 52 is disposed within the port 84 in the end cap 81 and prevents an enlarged portion, e.g., a ball 96, carried by a rod 94 engaged by the implant 95 to move distally through the port 84. In some instances, the cord 52 can extend distally of the ball 96, and in one or more embodiments, the cord 52 can terminate radially adjacent the ball 96. In one or more embodiments, the cross-sectional dimension of the ball 96 coupled with the cross-sectional dimension of the cord 52 is too large for the ball 96 to pass through the port 84. In such embodiments, the cord 52 and ball 96 obstruct the port by their engagement with one another proximally of the port 84. To detach the implant 95 from the positioner 40 at the implant interface 80, the cord 52 is moved in the proximal direction relative to the positioner tube 42 such that the distal tip 88 of the cord 52 is proximal of the port 84 in the end cap 81 and no longer obstructs the port 84. At this point, the ball 96 carried by the rod 94 and engaging the implant 95 is free to move distally through the port 84 or, alternatively, the positioner tube 42 or the entire positioner 40 can be moved in the proximal direction to allow the ball 96 to exit the positioner tube 42. Such configurations and methods are described in U.S. Patent Pub. No. 2010/0030200, the contents of which are hereby incorporated by reference to the extent not inconsistent with the present disclosure.

In yet further embodiments, detaching the implant 95 from the positioner 40 at the implant interface 80 can be realized through an electrolytic process. For instance, referring now to FIG. 2B, illustrated is another exemplary positioning system 100, according to one or more embodiments disclosed. The system 100 has a proximal end 102a and a distal end 102b and can include the implant 95, such as a coiled implant, arranged at or adjacent the distal end 102b. The system 100 can further include the positioner 40 extending from the user in conjunction with the implant interface 80. In one or more embodiments, the implant interface 80 can include a severance module 106 coupled to or otherwise arranged adjacent the implant 95. As illustrated, the system 100 can be a generally elongate structure having a long axis or longitudinal axis 110 where each of the implant 95, the severance module 106, and the positioner 40 are axially-offset along said longitudinal axis 110. The severance module 106 can require a voltage source and a cathode. The positioner 40 can include an insulating sleeve 116 shrink-fitted onto the outer surface of the positioner 40 and used, for example, to prevent the positioner 40 from corroding electrolytically. The implant 95 can serve as an anode and be slidably-arranged within the catheter. The severance module 106 has a severance location 120 that is electrolytically-corrodible so that when in contact with a bodily fluid or the like, the implant 95 will be detached by electrolytic processes.

As briefly described above, the positioner 40 and the implant 95 can be connected via the severance module 106 included in the implant interface 80. As will be appreciated, however, any connection that can be effectively detached from the implant 95 can be suitable for use as the implant interface 80. The severance module 106, for instance, can be suited for any kind of implant detachment or severance. For example, the severance module 106 can be designed for, but not limited to, mechanical, thermal, or electrochemical (e.g., electrolytic) detachment.

In one or more embodiments, the implant 95 can assume a predetermined, superimposed configuration after detachment. As used herein, the term “superimposed” refers to a shape or configuration that the implant 95 is configured to assume as preprogrammed through one or more heat treatment processes or methods undertaken by the strand 302. As discussed in more detail below, the superimposed configuration can include the implant 95 assuming a primary and/or a secondary shape.

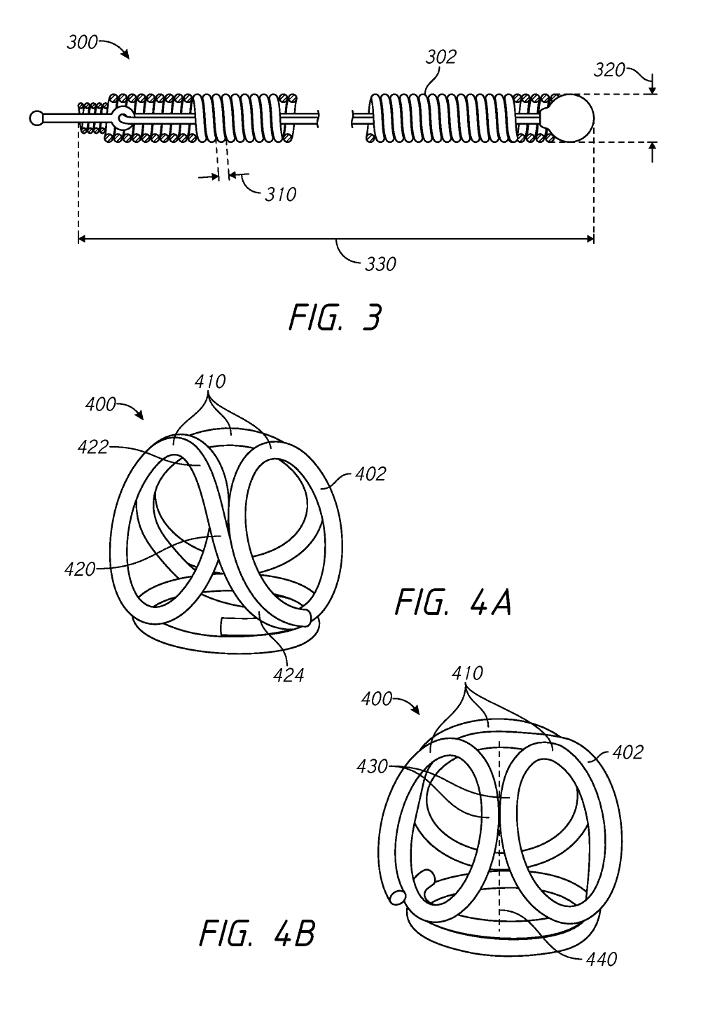

Referring to FIG. 3, in one or more embodiments, the implant 95 is made of a strand 302 that has been wound multiple times to form a generally tubular structure. In at least one embodiment, the strand 302 is wound so as to form a spiral helix, for example, a spiral helix forming several contiguous loops or windings having a pitch that is constant or alternatively varies over the length of the implant 95. As will be appreciated, however, the strand 302 can be formed or otherwise wound into several alternative configurations without departing from the scope of the disclosure.

In applications where the implant 95 is to be delivered to fine intracranial or cerebral vessels, implants having a coiled or spring structure can be particularly suited. As can be appreciated, the specific sizing of the implant 95 can be governed by the size of the treatment site or destination vessel and can be easily determined by those skilled in the art. In one or more embodiments, the primary shape of the implant 95 can have an outer diameter 320 that is between about 0.011 inch and about 0.0165 inch. By further example, in one or more embodiments, the primary shape of the implant 95 can have an outer diameter 320 that is between about 0.5 mm and about 10 mm. In other embodiments, however, the outer diameter 320 of the implant 95 can be less than about 0.5 mm and greater than about 10 mm, without departing from the scope of the disclosure. In one or more embodiments, the primary shape of the implant 95 can have a length that is between about 4 cm and about 65 cm.

In one or more embodiments, the strand 302 can be made of a nickel-titanium alloy (e.g., nitinol) which possesses both mechanical and thermal shape memory characteristics. In other embodiments, however, the strand 302 can be made of any material exhibiting mechanical and/or thermal shape memory characteristics or, alternatively, platinum, platinum alloys, tungsten, tungsten alloys, or other like materials. In one or more embodiments, the diameter 310 of the strand 302 can be between about 0.002 inch and about 0.004 inch. In one or more embodiments, the diameter 310 of the strand 302 can be between about 0.03 mm and about 0.3 mm. In other embodiments, the diameter 310 of the strand 302 can be between about 0.05 mm and about 0.2 mm. In at least one embodiment, the diameter 310 of the strand 302 can be about 0.06 mm. In yet other embodiments, the diameter 310 of the strand 302 can be from dimensions below about 0.03 mm and above about 0.3 mm, without departing from the scope of the disclosure.

In one or more embodiments, the strand 302 can be made of platinum or platinum alloys that have undergone a stress relief anneal process configured to help the strand 302 “remember” the superimposed or primary wound shape and automatically expand thereto. Strand 302 made of platinum and platinum alloys, or of similar materials, can also undergo stress relief annealing in order to better remember a secondary shape of the implant 95. In other embodiments the strand can be a nickel-titanium alloy and undergo a heat treatment configured to help the alloy remember a preprogrammed shape. Such a heat treatment can be comprised of restraining the strand in the desired shape, heat treating the restrained strand, then releasing the restraint. The implant 95 can achieve the preprogrammed shape in a relaxed state, in which the implant 95 is unrestrained, or the implant 95 can achieve the preprogrammed shape in an implanted state, in which the implant 95 is delivered to a target location with sufficient space to change its shape.

Referring now to FIGS. 4A-B, a coil, formed by a helically wound filament, can be formed into a secondary shape, according to one or more embodiments of the present disclosure. A “secondary” shape is formed using the primary shaped structure and creating a three-dimensional shape by, for example, wrapping the primary shaped structure around a mandrel and heat setting the primary shape in the wrapped disposition so the structure retains its primary coil shape as well as the secondary shape. As shown in FIG. 4A, an implant 400 can be formed of a primary coil 402 that transitions from a primary shape to a secondary shape, forming a plurality of loops 410 facing outwardly from a central region of the implant 400. Each of the loops 410 can lie within a plane and wind around an axis. In a relaxed state, the secondary shape of the implant 400 can form or conform to a three-dimensional polyhedral shape. The primary coil 402 can form a continuous structure that extends between and along adjacent and/or contiguous loops 410. Between contiguous loops 410, the primary coil 402 can transition at or along an inflection region 420 from a first arc shape 422 to a second arc shape 424. The first arc shape 422 can be convex with respect to a point in space, and the second arc shaped 424 can be concave with respect to the same point in space. Alternatively, the first arc shape 422 can be concave with respect to a point in space, and the second arc shaped 424 can be convex with respect to the same point in space. The transition along the inflection region 420 provides a continuous winding pattern that more uniformly distributes forces exerted by the resulting implant when conforming to body anatomy. As shown in FIG. 4B, pairs of loops 410 of the implant 400 can provide regions 430 that contact each other at or along a tangent line 440. Each of the loops 410 can contact and share tangent lines 440 with other loops 410. The adjacent regions 430 can separate or press against each other as the coil 400 transitions between shapes or is implanted and conforms to a target location.

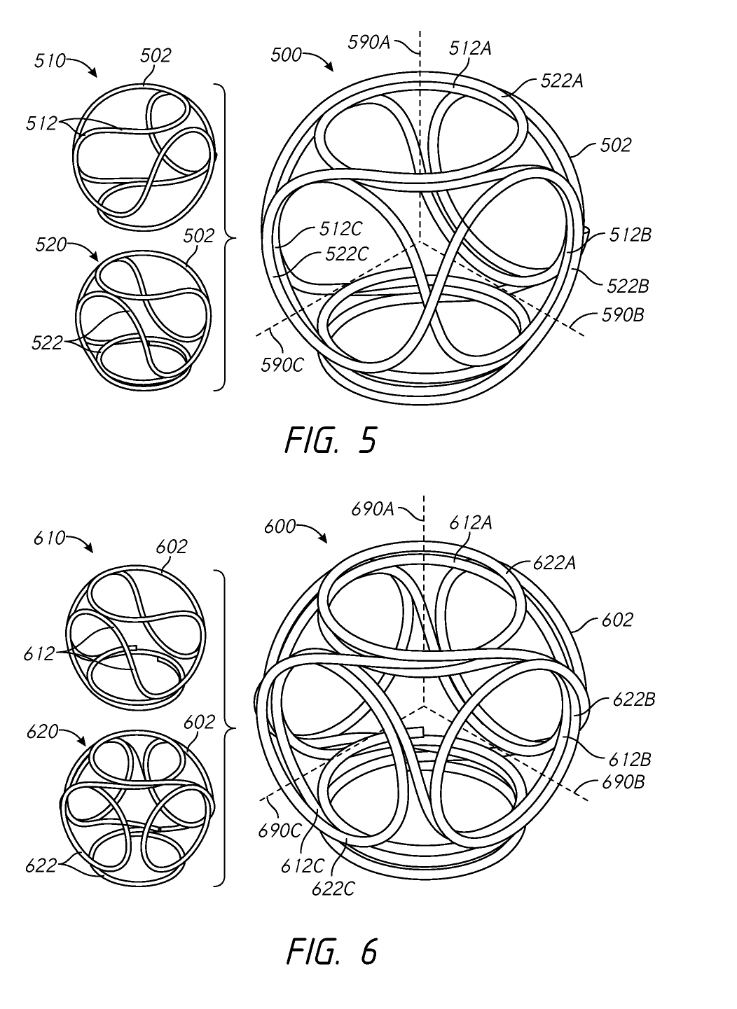

Referring now to FIG. 5, a continuous strand (e.g., forming a primary coil) can form a plurality of cycles, each cycle providing a plurality of loops to form a secondary shape with multiple layers, according to one or more embodiments of the present disclosure. As used herein, a “cycle” is a continuous formation that includes a plurality of loops, with only one loop about each axis of a plurality of axes, each loop comprising a continuous segment forming any number or fraction of revolutions about each axis of the plurality of axes. According to one or more embodiments, a continuous strand 502 (e.g., forming a primary coil) can form a plurality of cycles, such as a first cycle 510 and a second cycle 520. While the first cycle 510 and the second cycle 520 are shown separately in FIG. 5, both the first cycle 510 and the second cycle 520 can be formed by a single continuous strand 502. The first cycle 510 can include a plurality of first loops 512, with each first loop 512 winding about a corresponding axis, different from the axis of any other first loop 512. The second cycle 520 can include a plurality of second loops 522, with each second loop 522 winding about a corresponding axis, different from the axis of any other second loop 522. When formed of a continuous strand 502, the first cycle 510 and the second cycle 520 form overlapping first loops 512 and second loops 522. As shown in FIG. 5, each of the first loops 512a, 512b, and 512c of the first cycle 510 have corresponding axes 590a, 590b, and 590c. For each of these first loops 512a, 512b, and 512c, a corresponding one of the second loops 522a, 522b, and 522c of the second cycle 522 winds around the same axis (i.e., around an axis that is coaxial with the axis 590a, 590b, or 590c). Each of the first loops 512 can lie within a plane that is parallel to or coplanar with a plane of a corresponding one of the second loops 522. Each of the first loops 512 can be in contact or close proximity with a corresponding one of the second loops 522. For example, each first loop 512 can be radially adjacent to a corresponding second loop 522. The total number of first loops 512 in the first cycle 510 can be equal to the total number of second loops 522 in the second cycle 520.

According to one or more embodiments, loops of a cycle can be open loops or closed loops. As shown in FIG. 5, an implant 500 can form at least one first cycle 510 having a plurality of open loops 512 and at least one second cycle 520 having a plurality of open loops 522. As used herein, an “open loop” is a loop that does not overlap itself along a pathway about its axis (i.e., excluding sections of a strand that form any other loop or an inflection region transitioning to another loop). For example, an open loop can extend less than entirely (i.e., less than 360°) about a corresponding axis. By further example, each open loop forms an arc of an incomplete circle or other shape with ends that do not meet within the open loop. An exemplary open loop has an entire length that is separate from one or more adjacent loops. Each of the adjacent loops is wound about an axis that is different from the axis of the exemplary open loop. No part of the exemplary open loop overlaps, crosses, or intersects itself along the entire length thereof. As used herein, a “closed loop” is a loop that overlaps itself at least once along a pathway about its axis. For example, a closed loop can extend at least entirely (i.e., greater than or equal to 360°) about a corresponding axis. By further example, each closed loop forms a complete circle or other shape. An exemplary closed loop has an entire length that is separate from one or more adjacent loops. Each of the adjacent loops is wound about an axis that is different from the axis of the exemplary closed loop. At least part of the exemplary closed loop overlaps, crosses, or intersects itself along the entire length thereof.

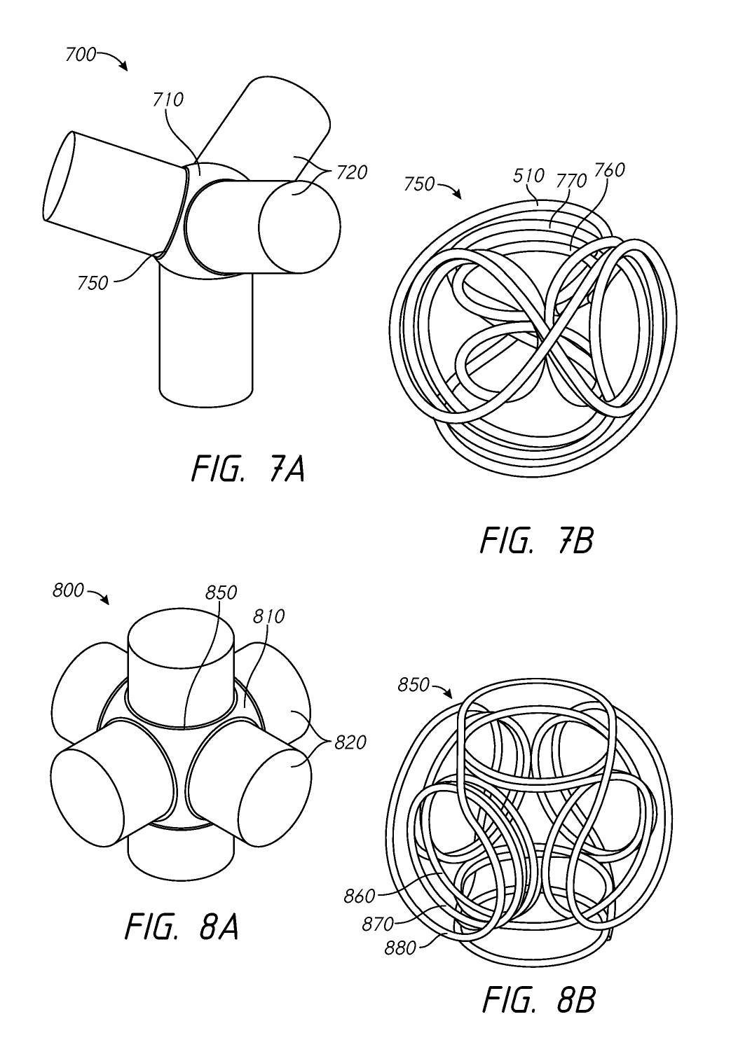

According to one or more embodiments, separate cycles can each have a distinct type of loop that is different from the type of loop (i.e., open or closed) in at least one other cycle. According to one or more embodiments, each and every one of the loops of a given cycle can be the same type of loop (i.e., open or closed). For example, as shown in FIG. 6, an implant 600 can include a strand 602 that forms at least one first cycle 610 and at least one second cycle 620 having a plurality of closed loops 622. Each of the loops 612 of the first cycle 610 can be open loops. Each of the loops 622 of the second cycle 620 can be closed loops. As shown in FIG. 6, each of the first loops 612a, 612b, and 612c of the first cycle 610 have corresponding axes 690a, 690b, and 690c. For each of these first loops 612a, 612b, and 612c, a corresponding one of the second loops 622a, 622b, and 622c of the second cycle 622 winds around the same axis (i.e., around an axis that is coaxial with the axis 690a, 690b, or 690c). Thus, each open loop can share an axis with a closed loop of a different cycle.

According to one or more embodiments, a closed loop provides a stiffer profile than that of an open loop. Accordingly, each closed loop will generate more force when deforming to fit within body anatomy and thereby generates a more stable structure to reduce movement and/or migration of the implant after delivery. According to one or more embodiments, an open loop provides greater flexibility and thereby promotes a greater degree of conformity with the body anatomy. By combining closed loops and open loops in different cycles of a secondary shape, an implant provides balanced stability and conformability upon delivery. The balance of stability and conformability can be modified by selecting combinations of open and closed loops.

According to one or more embodiments, a secondary shape of an implant can be provided with any number of cycles. For example, an implant can have, in a secondary shape, 2, 3, 4, 5, 6, 7, 8, 9, or 10 cycles. For any total number of cycles, at least one of the cycles can form only open loops and at least one of the cycles can form only closed loops. According to one or more embodiments, cycles can be formed by a continuous strand, such that each cycle contains contiguous loops that are not interrupted by the loops of any other cycle. Each cycle can terminate with ends at which additional cycles can begin or end. According to one or more embodiments, each cycle can be formed on a radially inner or radially outer surface of a different cycle. For example, at least one cycle can be fully encompassed or encapsulated by one or more other cycles. According to one or more embodiments, a radially innermost cycle of an implant forms only closed loops. According to one or more embodiments, a radially outermost cycle of an implant forms only closed loops. According to one or more embodiments, at least one cycle between a radially innermost cycle and a radially outermost cycle of an implant forms only open loops. According to one or more embodiments, an implant can include one or more additional loops adjacent to a radially innermost cycle and/or a radially outermost cycle. For example, additional loops can be used to secure the strand to a mandrel during a forming process.

Referring now to FIGS. 7A-B, a mandrel 700 can be employed to form an implant 750 having four loops in each cycle, according to one or more embodiments of the present disclosure. The mandrel 700 illustrated in FIG. 7A includes a core (e.g., sphere) 710 and four posts 720. The posts 720 are mounted on an external surface of the core 710 at four locations. Each of the posts 720 extends in a direction of a corresponding axis, wherein the axes extend into and/or intersect within the core 710. Each of the cycles of the implant 750 can be formed by winding a strand about each of the posts 720 once to form loops. The number of loops (e.g., four) of each cycle corresponds to the number of posts 720 of the mandrel 700. FIG. 7B illustrates an implant 750 formed with three cycles: a first cycle 760 forming only closed loops, a second cycle 770 forming only open loops, and a third cycle 780 forming only open loops. It will be appreciated that additional cycles and/or cycles having different types of loops can be formed using the mandrel 700 of FIG. 7A. The implant 750 of FIG. 7B forms a generally tetrahedral shape.

Referring now to FIGS. 8A-B, a mandrel 800 can be employed to form an implant 850 having six loops in each cycle, according to one or more embodiments of the present disclosure. The mandrel 800 illustrated in FIG. 8A includes a core (e.g., sphere) 810 and six posts 820. The posts 820 are mounted on an external surface of the core 810 at six locations. Each of the posts 820 extends in a direction of a corresponding axis, wherein the axes extend into and/or intersect within the core 810. Each of the cycles of the implant 850 can be formed by winding a strand about each of the posts 820 once to form loops. The number of loops (e.g., six) of each cycle corresponds to the number of posts 820 of the mandrel 800. FIG. 8B illustrates an implant 850 formed with three cycles: a first cycle 860 forming only closed loops, a second cycle 870 forming only open loops, and a third cycle 880 forming only open loops. It will be appreciated that additional cycles and/or cycles having different types of loops can be formed using the mandrel 800 of FIG. 8A. The implant 850 of FIG. 8B forms a generally hexahedral (e.g., cubic) shape.

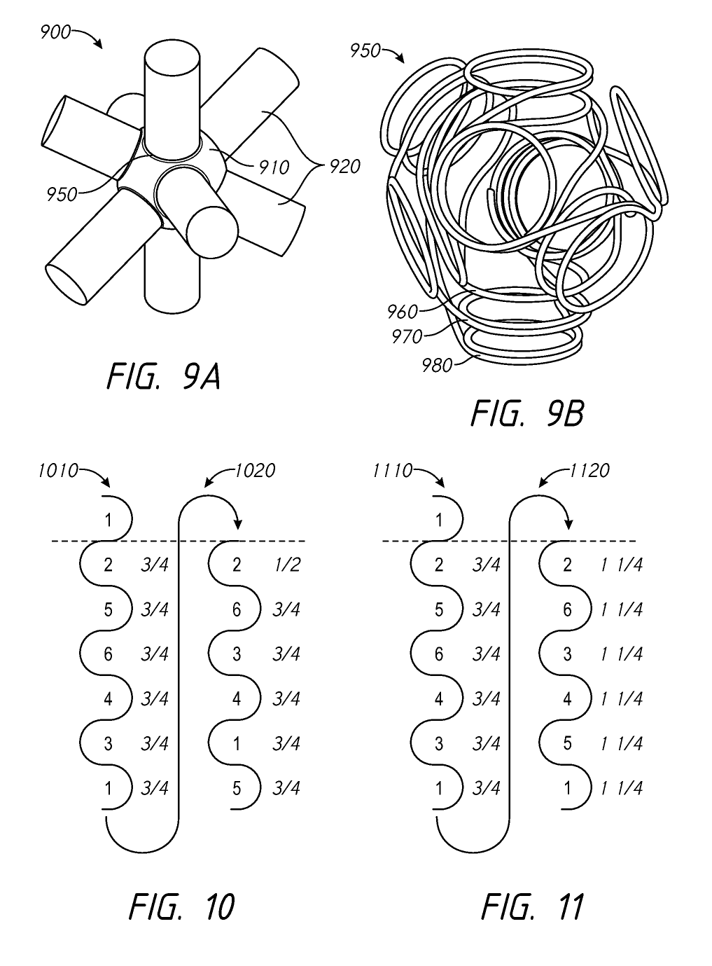

Referring now to FIGS. 9A-B, a mandrel 900 can be employed to form an implant 950 having eight loops in each cycle, according to one or more embodiments of the present disclosure. The mandrel 900 illustrated in FIG. 9A includes a core (e.g., sphere) 910 and eight posts 920. The posts 920 are mounted on an external surface of the core 910 at eight locations. Each of the posts 920 extends in a direction of a corresponding axis, wherein the axes extend into and/or intersect within the core 910. Each of the cycles of the implant 950 can be formed by winding a strand about each of the posts 920 once to form loops. The number of loops (e.g., eight) of each cycle corresponds to the number of posts 920 of the mandrel 900. FIG. 9B illustrates an implant 950 formed with three cycles: a first cycle 960 forming only open loops, a second cycle 970 forming only closed loops, and a third cycle 980 forming only closed loops. It will be appreciated that additional cycles and/or cycles having different types of loops can be formed using the mandrel 900 of FIG. 9A. The implant 950 of FIG. 9B forms a generally octahedral shape.

Referring now to FIGS. 10-11, winding patterns can provide the sequence and manner of winding about each of a number of posts of a mandrel, according to one or more embodiments of the present disclosure. FIG. 10 shows a first cycle 1010 and a second cycle 1020 as applied to a mandrel having six posts, such as the mandrel 800 of FIG. 8. As indicated at the top left corner of FIG. 10, the first cycle 1010 is preceded by a winding about a first post. The first cycle 1010 then provides partial winding (e.g., ¾ of a circumference) about each of the six posts to form open loops. The second cycle 1020 then provides partial winding (e.g., ½-¾ of a circumference) about each of the six posts to form open loops.

It will be appreciated that additional cycles and/or cycles having different types of loops can be formed using similar winding patterns. According to one or more embodiments, FIG. 11 shows a first cycle 1110 and a second cycle 1120 as applied to a mandrel having six posts, such as the mandrel 800 of FIG. 8. As indicated at the top left corner of FIG. 11, the first cycle 1110 is preceded by a winding about a first post. The first cycle 1110 then provides partial winding (e.g., ¾ of a circumference) about each of the six posts to form open loops. The second cycle 1120 then provides at least complete winding (e.g., 1¼ of a circumference) about each of the six posts to form closed loops. It will be appreciated that additional cycles and/or cycles having different types of loops can be formed using similar winding patterns.

With further reference to the winding patters of FIG. 10-11 and the exemplary mandrel and implant of FIG. 8, when forming the implant 850, the cycles are formed on the mandrel 800 and the wrapped implant 850 is subsequently subjected to a heat treatment process that causes the implant 850 to thereafter have a bias to the winding pattern about the mandrel 800, i.e., to have the predisposition to coil in a pattern similar to the winding pattern about the mandrel 800. After the implant 850 has been subjected to the heat treatment process, the implant 850 is removed from the mandrel 800.

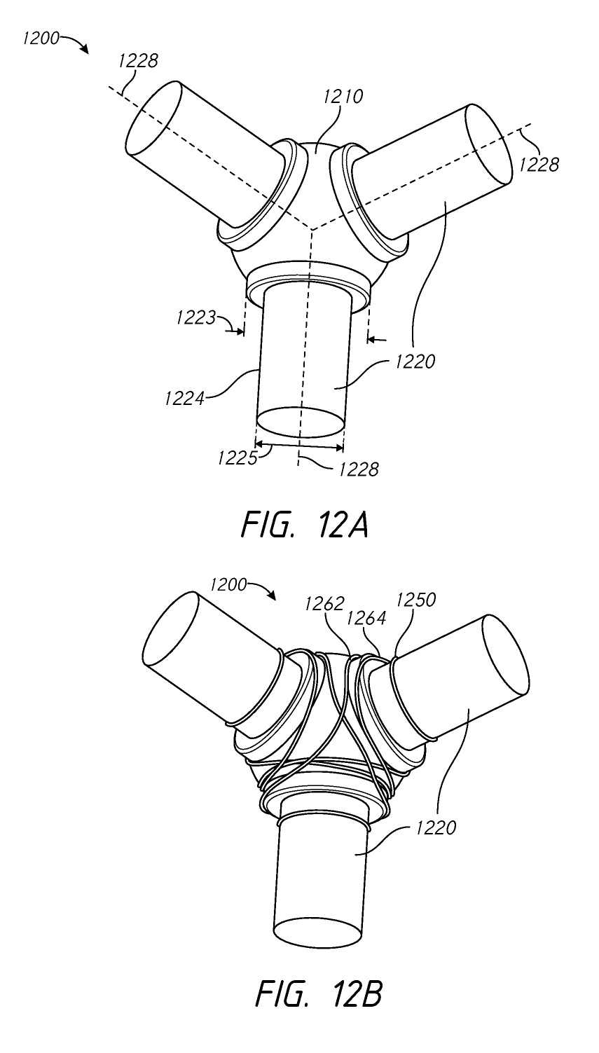

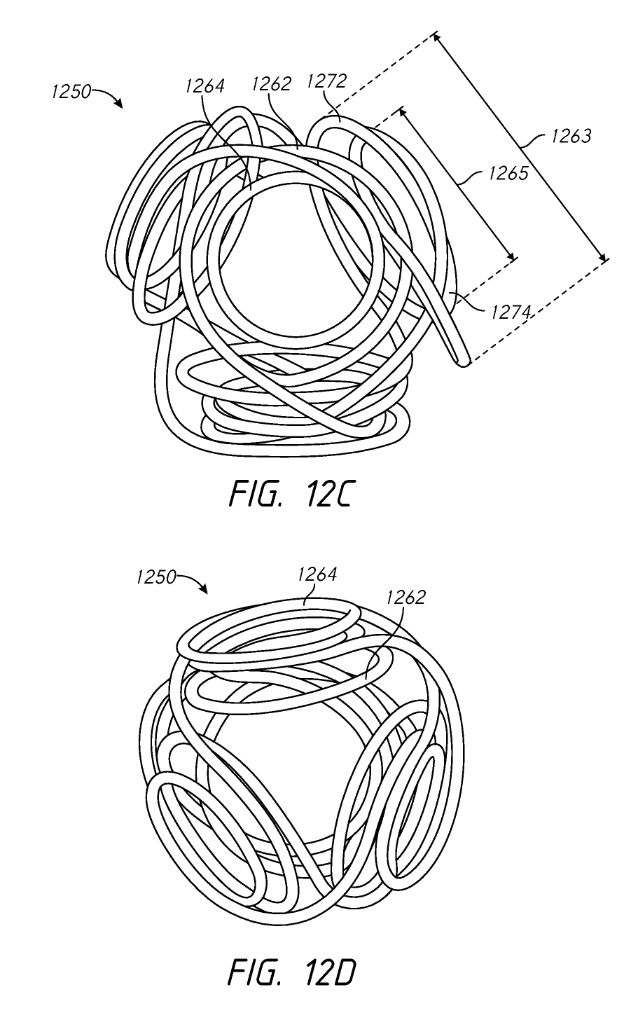

Referring now to FIGS. 12A-D, a mandrel 1200 can be employed to form an implant 1250 having different loop sizes in separate cycles, according to one or more embodiments of the present disclosure. The mandrel 1200 illustrated in FIG. 12A includes a core (e.g., sphere) 1210 and a plurality of posts 1220. Each post 1220 is mounted on an external surface of the core 1210 and extends in a direction of a corresponding axis 1228, wherein the axes 1228 extend into and/or intersect within the core 1210. One or more of the posts 1220 includes both a wider base section 1222 and a narrower extension section 1224. The base section 1222 can be closer to and/or adjacent to the core 1210 (i.e., between the core 1210 and the extension section 1224). The extension section 1224 can extend away from the corresponding base section 1222 and the core 1210. A base cross-sectional dimension 1223 (maximum or minimum) of the base section 1222 can be greater than an extension cross-sectional dimension 1225 (maximum or minimum) of the extension section 1224. As shown in FIG. 12A, the base section 1222 can have a constant cross-sectional dimension 1223 along a length thereof, and the extension section 1224 can have a constant cross-sectional dimension 1225 along a length thereof. The post 1220 can provide a stepwise transition from the base cross-sectional dimension 1223 to the extension cross-sectional dimension 1225. Alternatively, the posts 1220 can taper or otherwise gradually change cross-sectional dimension along the lengths thereof, extending away from the core 1210. It will be appreciated that additional sections of each post 1220 can be provided with distinct cross-sectional dimensions. For example, each post 1220 can include 3, 4, 5, 6, 7, or more than 7 sections with distinct cross-sectional dimensions.

FIG. 12B shows the implant 1250 formed on the mandrel 1200. A first cycle 1262 and a second cycle 1264 of the implant 1250 can each be formed by winding a strand about each of the posts 1220 once to form loops. For example, the first cycle 1262 can be formed by winding a strand once about the base section 1222 of each post 1220, and the second cycle 1264 can be formed by winding the strand once about the extension section 1224 of each post 1220. The loops 1272 of the first cycle 1262 can be open or closed loops, and the loops 1274 of the second cycle 1264 can be open or closed loops. For example, the loops 1272 of the first cycle 1262 can be closed loops to provide more structural stability than would be provided by open loops. The closed loops can be formed where the loops have a larger cross-sectional dimension than the cross-sectional dimension of loops of at least one other cycle. When forming the implant 1250, the cycles are formed on the mandrel 1200, and the wrapped implant 1250 is subsequently subjected to a heat treatment process that causes the implant 1250 to thereafter have a bias to a secondary shape according to the winding pattern about the mandrel 1200. After the implant 1250 has been subjected to the heat treatment process, the implant 1250 is removed from the mandrel 1200.

As shown in FIGS. 12C-D, the cross-sectional dimension of the resulting loops correspond to the cross-sectional dimensions of the posts 1220 of the mandrel 1200. For example, the loops 1272 of the first cycle 1262 can each have a cross-sectional dimension 1263 that is substantially equal to the cross-sectional dimension 1223 of the base section 1222, and the loops 1274 of the second cycle 1264 can each have a cross-sectional dimension 1265 that is substantially equal to the cross-sectional dimension 1225 of the extension section 1224. Accordingly, the cross-sectional dimensions 1263 (maximum or minimum) of the loops 1272 of the first cycle 1262 can be greater than the cross-sectional dimensions 1265 (maximum or minimum) of the loops 1274 of the second cycle 1264. Each of the loops 1272 of the first cycle 1262 can lie within a plane that is parallel to and/or non-intersecting with a plane of a corresponding one of the loops 1274 of the second cycle 1264. Each of the loops 1272 of the first cycle 1262 can share a common central axis with a corresponding one of the loops 1274 of the second cycle 1264. It will be appreciated that additional cycles and/or cycles having different types of loops can be formed using the mandrel 1200 of FIG. 12A.

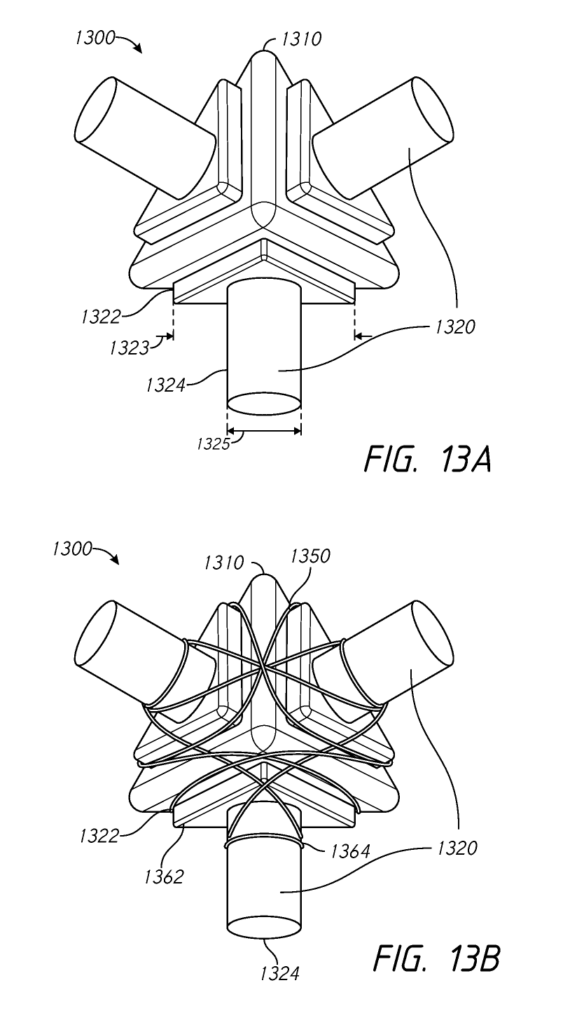

Referring now to FIGS. 13A-C, a mandrel 1300 can be employed to form an implant 1350 having different loop shapes in separate cycles, according to one or more embodiments of the present disclosure. The mandrel 1300 illustrated in FIG. 13A includes a core (e.g., polyhedron) 1310 and a plurality of posts 1320. Each post 1320 is mounted on an external surface of the core 1310 and extends in a direction of a corresponding axis, wherein the axes extend into and/or intersect within the core 1310. One or more of the posts 1320 includes both a base section 1322 and an extension section 1324. The base section 1322 can be closer to and/or adjacent to the core 1310 (i.e., between the core 1310 and the extension section 1324). The extension section 1324 can extend away from the corresponding base section 1322 and the core 1310. The base section 1322 and the extension section 1324 can have different cross-sectional or on-face shapes. A cross-sectional shape of a section can be defined in a cross-section orthogonal to an axis. Different sections can have different cross-sectional shapes defined by separate cross-sections along the same axis or parallel axes. An on-face shape of a section can be defined in a view along an axis. Different sections can have different on-face shapes defined by views along the same axis or parallel axes. As used herein, different shapes do not include same or similar shapes scaled to different sizes. Rather, different shapes indicate shapes that are different in at least one respect other than size. The shapes of each of the base section 1322 and the extension section 1324 can be, for example, a shape of a circle, oval, ellipse, polygon, triangle, square, pentagon, hexagon, quadrilateral, star, or another geometric shape. It will be appreciated that additional sections of each post 1320 can be provided with distinct shapes. For example, each post 1320 can include 3, 4, 5, 6, 7, or more than 7 sections with distinct shapes. For example, as shown in FIG. 13A, the base section 1322 can have a polygonal (e.g., triangular) shape, and the extension section 1324 can have a circular shape. A base cross-sectional dimension 1323 (maximum or minimum) of the base section 1322 can be greater than an extension cross-sectional dimension 1325 (maximum or minimum) of the extension section 1324. A minimum base cross-sectional dimension 1323 can be greater than or equal to a maximum extension cross-sectional dimension 1325. As shown in FIG. 13A, the base section 1322 can have a constant shape and cross-sectional dimension 1323 along a length thereof, and the extension section 1324 can have a constant shape and cross-sectional dimension 1325 along a length thereof. The post 1320 can provide a stepwise transition from the base shape and cross-sectional dimension 1323 to the extension shape and cross-sectional dimension 1325. Alternatively, the posts 1320 can taper or otherwise gradually change shape and cross-sectional dimension along the lengths thereof, extending away from the core 1310.

FIG. 13B shows the implant 1350 formed on the mandrel 1300. A first cycle 1362 and a second cycle 1364 of the implant 1350 can each be formed by winding a strand about each of the posts 1320 once to form loops. For example, the first cycle 1362 can be formed by winding a strand once about the base section 1322 of each post 1320, and the second cycle 1364 can be formed by winding the strand once about the extension section 1324 of each post 1320. The loops 1372 of the first cycle 1362 can be open or closed loops, and the loops 1374 of the second cycle 1364 can be open or closed loops. For example, the loops 1372 of the first cycle 1362 can be closed loops to provide more structural stability than would be provided by open loops. The closed loops can be formed where the loops have a larger cross-sectional dimension than the cross-sectional dimension of loops of at least one other cycle. When forming the implant 1350, the cycles are formed on the mandrel 1300, and the wrapped implant 1350 is subsequently subjected to a heat treatment process that causes the implant 1350 to thereafter have a bias to a secondary shape according to the winding pattern about the mandrel 1300. After the implant 1350 has been subjected to the heat treatment process, the implant 1350 is removed from the mandrel 1300.

As shown in FIG. 13C, the cross-sectional dimension of the resulting loops correspond to the cross-sectional dimensions of the posts 1320 of the mandrel 1300. For example, the loops 1372 of the first cycle 1362 can each have a shape and cross-sectional dimension 1363 that is substantially equal to the shape and cross-sectional dimension 1323 of the base section 1322, and the loops 1374 of the second cycle 1364 can each have a shape and cross-sectional dimension 1365 that is substantially equal to the shape and cross-sectional dimension 1325 of the extension section 1324. Accordingly, the cross-sectional dimensions 1363 (maximum or minimum) of the loops 1372 of the first cycle 1362 can be greater than the cross-sectional dimensions 1365 (maximum or minimum) of the loops 1374 of the second cycle 1364, and the shape of the loops 1372 of the first cycle 1362 can be different from the shape of the loops 1374 of the second cycle 1364. Each of the loops 1372 of the first cycle 1362 can lie within a plane that is parallel to and/or non-intersecting with a plane of a corresponding one of the loops 1374 of the second cycle 1364. Each of the loops 1372 of the first cycle 1362 can share a common central axis with a corresponding one of the loops 1374 of the second cycle 1364. It will be appreciated that additional cycles and/or cycles having different types of loops can be formed using the mandrel 1300 of FIG. 13A.

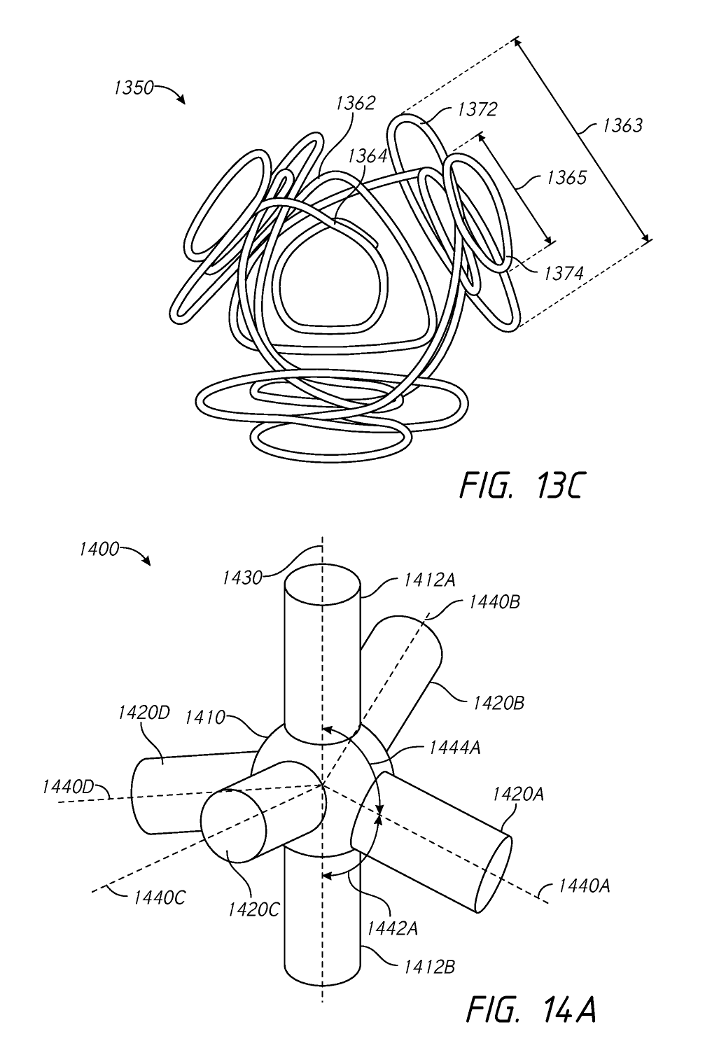

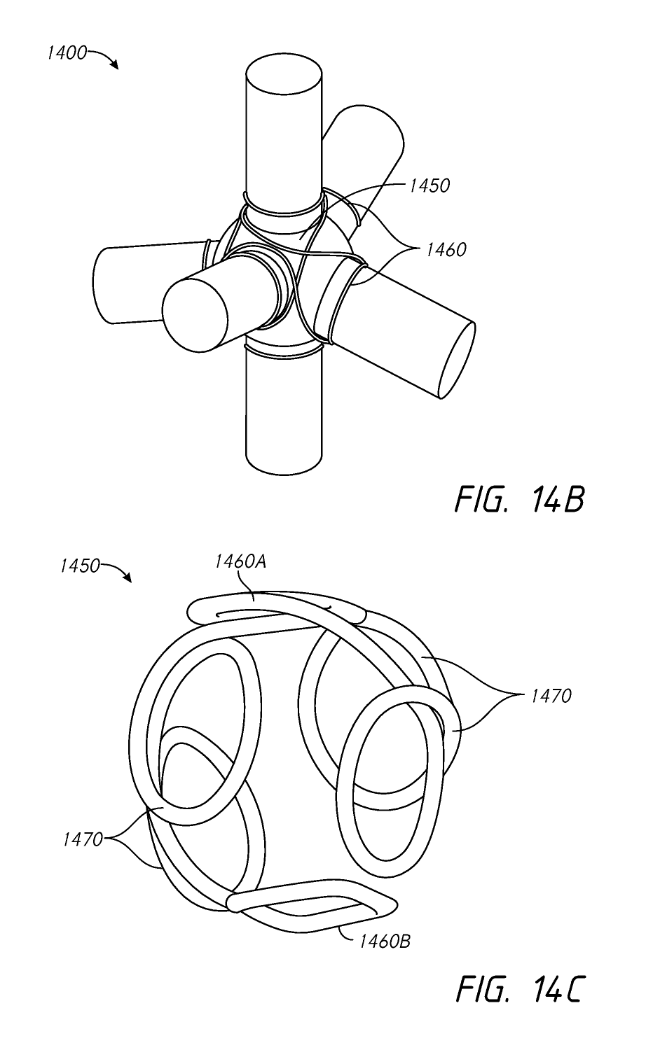

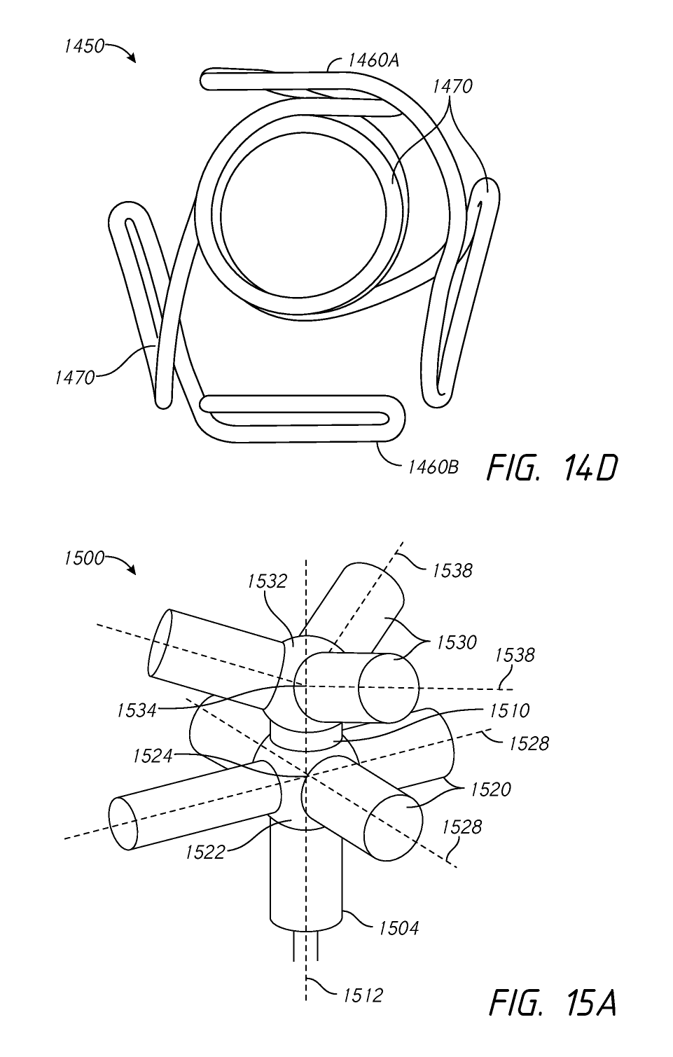

Referring now to FIGS. 14A-D, a mandrel 1400 can be employed to form an implant 1450 having loops with non-uniform distribution, according to one or more embodiments of the present disclosure. The mandrel 1400 illustrated in FIG. 14A includes a core (e.g., sphere) 1410 and a plurality of primary posts 1412a and 1412b and a plurality of secondary posts 1420a, 1420b, 1420c, and 1420d. Each of the primary posts 1412a and 1412b is mounted on an external surface of the core 1410 and extends in a direction of the primary axis 1430, wherein the primary axis 1430 extends into the core 1410. The primary posts 1412a and 1412b can be coaxial, such that each of the primary posts 1412a and 1412b extends along the primary axis 1430 on opposite sides of the core 1410. Each of the secondary posts 1420a, 1420b, 1420c, and 1420d is mounted on an external surface of the core 1410 and extends in a direction of a corresponding secondary axis 1440a, 1440b, 1440c, or 1440d. The secondary axes 1440a, 1440b, 1440c, and 1440d extend into the core 1410 and intersect with each other and/or the primary axis 1430. Each of the secondary posts 1420a, 1420b, 1420c, and 1420d can extend along a unique axis, such that none of the secondary posts 1420a, 1420b, 1420c, and 1420d is coaxial with any other one of the secondary posts 1420a, 1420b, 1420c, and 1420d. Each of the secondary axes 1440a, 1440b, 1440c, and 1440d can intersect with the primary axis 1430. As shown in FIG. 14A, the secondary axis 1440a of the secondary post 1420a can intersect with the primary axis 1430 to form a minor angle 1442a and a major angle 1444a. The minor angle 1442a can be different from the major angle 1444a, such that the secondary post 1420a is oriented in a direction that is transverse to the primary axis 1430 and is closer to one of the primary posts 1412a and 1412b than the other of the primary posts 1412a and 1412b. The angles described above with respect to the secondary post 1420a can apply to one or more of the secondary posts 1420a, 1420b, 1420c, and 1420d. Some of the secondary posts 1420a, 1420b, 1420c, and 1420d can be closer to one of the primary posts 1412a and 1412b and others of the secondary posts 1420a, 1420b, 1420c, and 1420d can be closer to the other of the primary posts 1412a and 1412b. The minor angles of each of the secondary posts 1420a, 1420b, 1420c, and 1420d can be equal to each other and the major angles of each of the secondary posts 1420a, 1420b, 1420c, and 1420d can be equal to each other. While the mandrel 1400 of FIG. 14A is shown having four secondary posts, it will be appreciated that any number of secondary posts with corresponding angular orientations can be provided. For example, the mandrel 1400 can include 1, 2, 3, 4, 5, 6, 7, 8, 9, 10 or more than 10 secondary posts.

FIG. 14B shows the implant 1450 formed on the mandrel 1400. While the implant 1450 is shown having only one cycle, it will be appreciated that multiple cycles can be formed, as further described herein. The loops of each cycle can be open or closed loops. When forming the implant 1450, the cycles are formed on the mandrel 1400, and the wrapped implant 1450 is subsequently subjected to a heat treatment process that causes the implant 1450 to thereafter have a bias to a secondary shape according to the winding pattern about the mandrel 1400. After the implant 1450 has been subjected to the heat treatment process, the implant 1450 is removed from the mandrel 1400.

As shown in FIGS. 14C-D, the distribution of primary loops 1460a and 1460b and secondary loops 1470 corresponds to the locations and orientations of the primary posts 1412a and 1412b and secondary posts 1420a, 1420b, 1420c, and 1420d of the mandrel 1400. For example, some secondary loops 1470 are closer to the first primary loop 1460a than they are close to the second primary loop 1460b, and other secondary loops 1470 are closer to the second primary loop 1460b than they are close to the first primary loop 1460a. Accordingly, the forces exerted by the implant 1450 in various directions are asymmetric because at least some of the secondary loops 1470 are not balanced by another secondary loop 1470 on a diametrically opposite side of the implant 1450. The resulting distribution allows each of the secondary loops 1470, when implanted, to provide a force that is not balanced by another secondary loop 1470. Such configurations can be provided and/or adapted to accommodate aneurysms of irregular shapes. The asymmetric distribution of secondary loops 1470 allows the implant 1450 to conform to an irregular shape with a balanced force distribution when implanted in such an irregular shape. It will be appreciated that various winding patterns and cycles can be employed to form an implant 1450 using the mandrel 1400 of FIG. 14A.

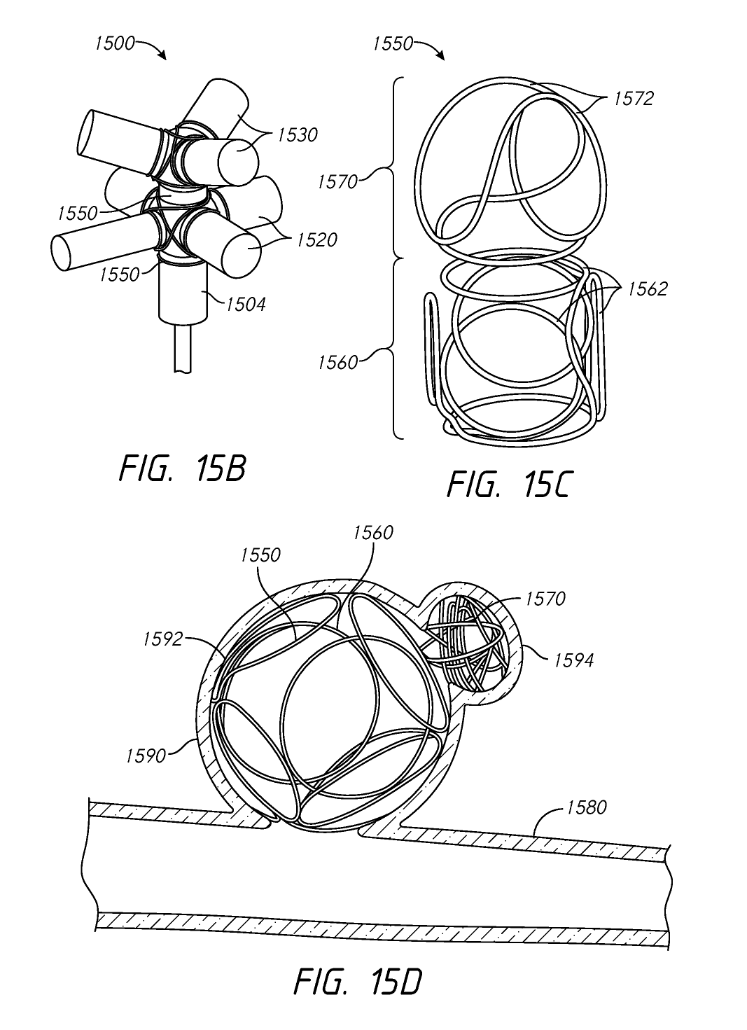

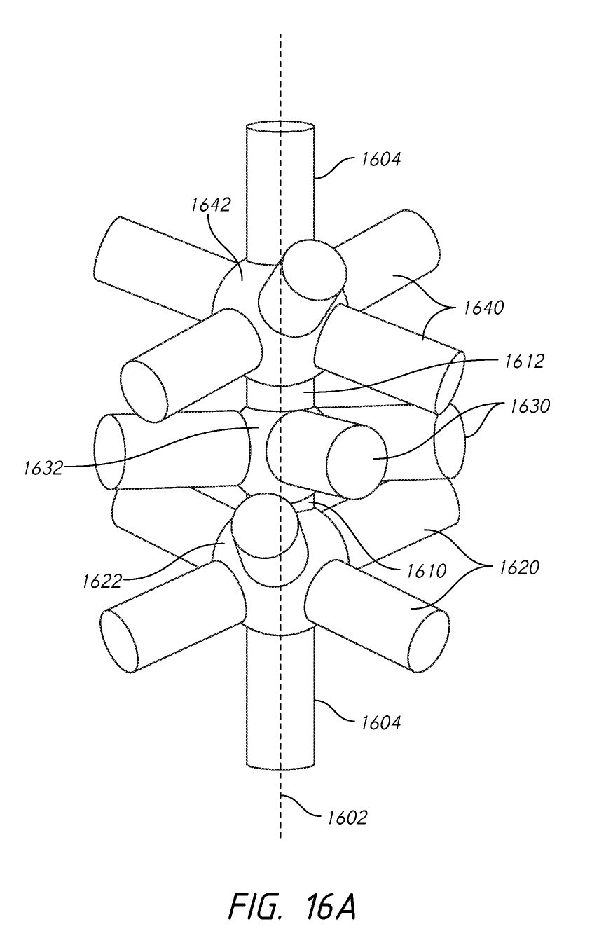

Referring now to FIGS. 15A-D, a mandrel 1500 can be employed to form an implant 1550 having multiple sections, according to one or more embodiments of the present disclosure. The mandrel 1500 illustrated in FIG. 15A includes a first core (e.g., sphere) 1522 and a second core (e.g., sphere) 1532. One or more main posts 1504 can extend along a main axis 1512. A plurality of first posts 1520 are mounted on an external surface of the first core 1522 and each extend in a direction of a corresponding first axis 1528, wherein the first axes 1528 each extend into the first core 1522. Some or all of the first axes 1528 can intersect at a first central region 1524. A plurality of second posts 1530 are mounted on an external surface of the second core 1532 and each extend in a direction of a corresponding second axis 1538, wherein the second axes 1538 each extend into the second core 1532. Some or all of the first axes 1538 can intersect at a second central region 1534. The first central region and the second central region can be aligned along the main axis 1512 that extends through the first core 1522 and the second core 1532. At least one common post 1510 can extend between the first core 1522 and the second core 1532, such that a winding patter about each of the first core 1522 and the second core 1532 can include contact with the common post 1510. A total number of first posts 1520 can be equal to, greater than, or less than a total number of second posts 1530. A diameter or cross-sectional dimension of the first core 1522 can be equal to, larger than, or smaller than a diameter or cross-sectional dimension of the second core 1532. A size or cross-sectional shape of the first posts 1520 can be the same as or different from a size or cross-sectional shape of the second posts 1530. Some or all of the first axes 1528 can be orthogonal to the main axis 1512, and some or all of the second axes 1538 can be transverse to the main axis 1512. While the mandrel 1500 of FIG. 15A is shown having five first posts 1520 and three secondary posts 1530, it will be appreciated that any number of first posts 1520 and secondary posts 1530 with corresponding orientations can be provided. For example, each core and corresponding posts can be provided in accordance with the disclosure provided with regard to mandrel 700, mandrel 800, mandrel 900, mandrel 1200, mandrel 1300, and/or mandrel 1400.

FIG. 15B shows the implant 1550 formed on the mandrel 1500. While the implant 1550 is shown having only one cycle, it will be appreciated that multiple cycles can be formed, as further described herein. Each cycle can extend across both of the first core 1522 and the second core 1532. Alternatively, a plurality of cycles can extend across the first core 1522 and another plurality of cycles can extend across the second core 1532. The loops of each cycle can be open or closed loops. When forming the implant 1550, the cycles are formed on the mandrel 1500, and the wrapped implant 1550 is subsequently subjected to a heat treatment process that causes the implant 1550 to thereafter have a bias to a secondary shape according to the winding pattern about the mandrel 1500. After the implant 1550 has been subjected to the heat treatment process, the implant 1550 is removed from the mandrel 1500.

As shown in FIG. 15C, the implant 1550 formed as described herein can provide a first section 1560, corresponding to a winding about the first core 1522 and first posts 1528, and a second section 1570, corresponding to a winding about the second core 1532 and second posts 1538. For example, first loops 1562 can each have a shape and cross-sectional dimension that is substantially equal to the shape and cross-sectional dimension of the first posts 1528, and second loops 1572 can each have a shape and cross-sectional dimension that is substantially equal to the shape and cross-sectional dimension of the second posts 1538. A total number of first loops 1562 can be equal to, greater than, or less than a total number of second loops 1572. A diameter or cross-sectional dimension of the first section 1560 can be equal to, larger than, or smaller than a diameter or cross-sectional dimension of the second section 1570. A size or cross-sectional shape of the first loops 1562 can be the same as or different from a size or cross-sectional shape of the second loops 1572. It will be appreciated that any number of first loops 1562 and second loops 1572 with corresponding orientations can be provided. For example, each section and set of loops can be provided in accordance with the disclosure provided with regard to implant 95, implant 400, implant 500, implant 600, implant 750, implant 850, implant 950, implant 1250, implant 1350, and/or implant 1450.

As shown in FIG. 15D, the implant 1550 formed as described herein can be implanted within an aneurysm 1590 having a first region 1592 and a second region 1594 extending from a vessel 1580. The implant 1550 can be implanted such that the first section 1560 thereof is positioned within the first region 1592 of the aneurysm 1590 and the second section 1570 is positioned within the second region 1594 of the aneurysm 1590. Each of the first section 1560 and the second section 1570 can be sized and shaped to substantially conform to the first region 1592 and the second region 1594, respectively.

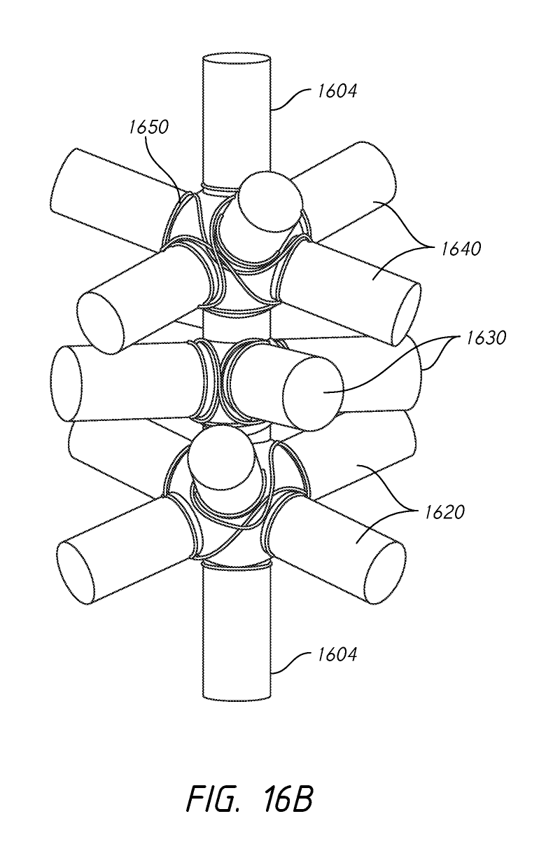

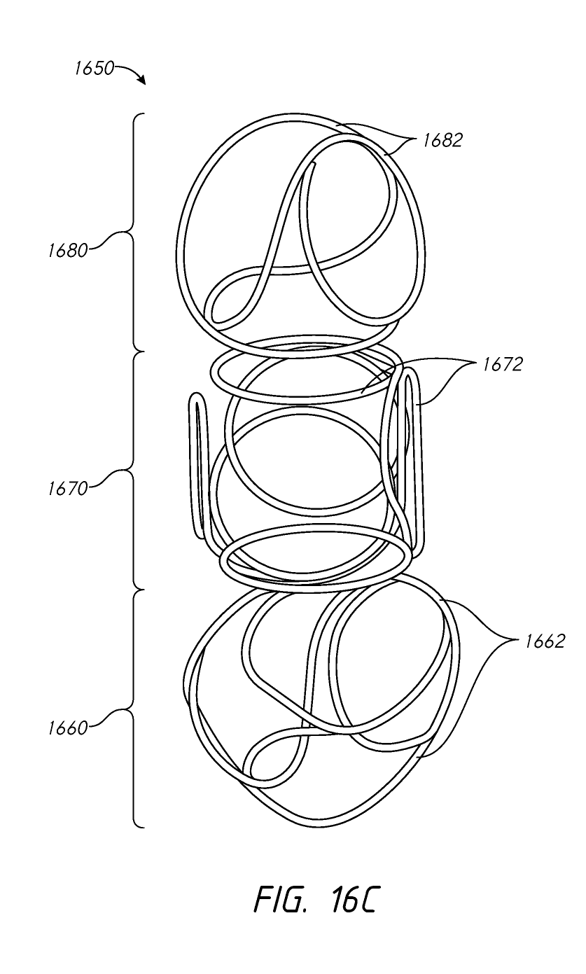

Referring now to FIGS. 16A-C, a mandrel 1600 can be employed to form an implant 1650 having multiple sections, according to one or more embodiments of the present disclosure. The mandrel 1600 illustrated in FIG. 16A includes a first core (e.g., sphere) 1622, a second core (e.g., sphere) 1632, and a third core (e.g., sphere) 1642. One or more main posts 1604 can extend along a main axis 1602. A plurality of first posts 1620 are mounted on an external surface of the first core 1622, a plurality of second posts 1630 are mounted on an external surface of the second core 1632, and a plurality of third posts 1640 are mounted on an external surface of the third core 1642. The first core 1622, the second core 1632, and the third core 1642 can be aligned along the main axis 1602. At least one common post 1610 can extend between the first core 1622 and the second core 1632, such that a winding patter about each of the first core 1622 and the second core 1632 can include contact with the common post 1610. At least one common post 1612 can extend between the second core 1632 and the third core 1642, such that a winding patter about each of the second core 1632 and the third core 1642 can include contact with the common post 1612. Each core and its corresponding posts can be provided in accordance with the disclosure provided with regard to mandrel 700, mandrel 800, mandrel 900, mandrel 1200, mandrel 1300, mandrel 1400, and/or mandrel 1500. It will be appreciated that any number of first posts 1620, secondary posts 1630, and third posts 1640 can be provided. Some or all of the first posts 1620 can be transverse to the main axis 1602, some or all of the second posts 1630 can be orthogonal to the main axis 1602, and some or all of the third posts 1640 can be transverse to the main axis 1602.

A total number of first posts 1620 can be equal to, greater than, or less than a total number of second posts 1630. A diameter or cross-sectional dimension of the first core 1622 can be equal to, larger than, or smaller than a diameter or cross-sectional dimension of the second core 1632. A size or cross-sectional shape of the first posts 1620 can be the same as or different from a size or cross-sectional shape of the second posts 1630.

A total number of second posts 1630 can be equal to, greater than, or less than a total number of third posts 1640. A diameter or cross-sectional dimension of the second core 1632 can be equal to, larger than, or smaller than a diameter or cross-sectional dimension of the third core 1642. A size or cross-sectional shape of the second posts 1630 can be the same as or different from a size or cross-sectional shape of the third posts 1640.

A total number of first posts 1620 can be equal to, greater than, or less than a total number of third posts 1640. A diameter or cross-sectional dimension of the first core 1622 can be equal to, larger than, or smaller than a diameter or cross-sectional dimension of the third core 1642. A size or cross-sectional shape of the first posts 1620 can be the same as or different from a size or cross-sectional shape of the third posts 1640.

FIG. 16B shows the implant 1650 formed on the mandrel 1600. While the implant 1650 is shown having only one cycle, it will be appreciated that multiple cycles can be formed, as further described herein. Each cycle can extend across both of the first core 1622, the second core 1632, and the third core 1642. Alternatively, a plurality of cycles can extend across the first core 1622, a plurality of cycles can extend across the second core 1632, and a plurality of cycles can extend across the third core 1642. The loops of each cycle can be open or closed loops. When forming the implant 1650, the cycles are formed on the mandrel 1600, and the wrapped implant 1650 is subsequently subjected to a heat treatment process that causes the implant 1650 to thereafter have a bias to a secondary shape according to the winding pattern about the mandrel 1600. After the implant 1650 has been subjected to the heat treatment process, the implant 1650 is removed from the mandrel 1600.

As shown in FIG. 16C, the implant 1650 formed as described herein can provide a first section 1660, corresponding to a winding about the first core 1622 and first posts 1628, a second section 1670, corresponding to a winding about the second core 1632 and second posts 1638, and a third section 1680, corresponding to a winding about the third core 1642 and third posts 1648. For example, first loops 1662 can each have a shape and cross-sectional dimension that is substantially equal to the shape and cross-sectional dimension of the first posts 1628, second loops 1672 can each have a shape and cross-sectional dimension that is substantially equal to the shape and cross-sectional dimension of the second posts 1638, and third loops 1682 can each have a shape and cross-sectional dimension that is substantially equal to the shape and cross-sectional dimension of the third posts 1648.

A total number of first loops 1662 can be equal to, greater than, or less than a total number of second loops 1672. A diameter or cross-sectional dimension of the first section 1660 can be equal to, larger than, or smaller than a diameter or cross-sectional dimension of the second section 1670. A size or cross-sectional shape of the first loops 1662 can be the same as or different from a size or cross-sectional shape of the second loops 1672.

A total number of second loops 1672 can be equal to, greater than, or less than a total number of third loops 1682. A diameter or cross-sectional dimension of the second section 1670 can be equal to, larger than, or smaller than a diameter or cross-sectional dimension of the third section 1680. A size or cross-sectional shape of the second loops 1672 can be the same as or different from a size or cross-sectional shape of the third loops 1682.

A total number of first loops 1662 can be equal to, greater than, or less than a total number of third loops 1682. A diameter or cross-sectional dimension of the first section 1660 can be equal to, larger than, or smaller than a diameter or cross-sectional dimension of the third section 1680. A size or cross-sectional shape of the first loops 1662 can be the same as or different from a size or cross-sectional shape of the third loops 1682.

It will be appreciated that any number of first loops 1662, second loops 1672, and third loops 1682 can be provided. Each section and set of loops can be provided in accordance with the disclosure provided with regard to implant 95, implant 400, implant 500, implant 600, implant 750, implant 850, implant 950, implant 1250, implant 1350, implant 1450, and/or implant 1550.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

I/We claim:

1. An embolic implant comprising:
a strand forming, in a relaxed state, (i) first consecutive loops in a first cycle along a first length of the strand and (ii) second consecutive loops in a second cycle along a second length of the strand;
wherein each of the first loops is an open loop that extends partially about a corresponding first axis, and each of the second loops is a closed loop that extends completely about a corresponding second axis that extends through a space within a radially adjacent one of the first loops.

2. The embolic implant of claim 1, wherein the strand forms, in the relaxed state, a three-dimensional polyhedral shape.

3. The embolic implant of claim 1, wherein, in the relaxed state, each of the first loops contacts and shares tangent lines with at least one other first loop.

4. The embolic implant of claim 1, wherein a total number of the first loops is equal to a total number of the second loops.

5. The embolic implant of claim 1, wherein the strand further forms, in the relaxed state, consecutive third loops in a third cycle along a third length of the strand, each of the third loops extends about a corresponding third axis that that extends through a space within a radially adjacent one of the second loops.

6. The embolic implant of claim 5, wherein each of the third loops is an open loop that extends partially about the third axis.

7. The embolic implant of claim 5, wherein each of the third loops is a closed loop that extends completely about the third axis.

8. The embolic implant of claim 1, wherein the first consecutive loops form a radially innermost section of the implant.

9. The embolic implant of claim 1, wherein the second consecutive loops form a radially outermost section of the implant.

10. A method of forming an embolic implant, the method comprising:
winding a first length of a strand in a first cycle partially about each of a plurality of posts extending from a core of a mandrel to form consecutive open first loops; and
winding a second length of the strand in a second cycle completely about each of the posts to form consecutive closed second loops.

11. The method of claim 10, wherein the first length and the second length are wound such that the strand forms a three-dimensional polyhedral shape.

12. The method of claim 10, wherein the first length is wound such that each of the first loops contacts and shares tangent lines with other first loops.

13. The method of claim 10, wherein the second length is wound to form a total number of the second loops equal to a total number of the first loops.

14. The method of claim 10, further comprising winding a third length of the strand in a third cycle about each of the posts to form consecutive third loops.

15. The method of claim 14, wherein the third length is wound such that each of the third loops is an open loop that extends partially about a corresponding one of the posts.

16. The method of claim 14, wherein the third length is wound such that each of the third loops is a closed loop that extends completely about a corresponding one of the posts.

17. A method of delivering an embolic implant, the method comprising:
providing an implant within a delivery device to a target location while the implant is in a first, substantially straight configuration; and
positioning the implant at the target location such that the implant is in a secondary configuration in which the implant comprises:
a strand forming (i) first consecutive loops in a first cycle along a first length of the strand and (ii) second consecutive loops in a second cycle along a second length of the strand;
wherein each of the first loops is an open loop that extends partially about a corresponding first axis, and each of the second loops is a closed loop that extends completely about a corresponding second axis that that extends through a space within a radially adjacent one of the first loops.

18. The method of claim 17, wherein the implant is positioned to form, in the secondary configuration, a three-dimensional polyhedral shape.

19. The method of claim 17, wherein the implant is positioned, in the secondary configuration, such that each of the first loops contacts and shares tangent lines with other first loops.

20. The method of claim 17, wherein the implant is positioned, in the secondary configuration, such that a total number of the first loops is equal to a total number of the second loops.

21. The method of claim 17, wherein the implant is positioned, in the secondary configuration, to form consecutive third loops in a third cycle along a third length of the strand, each of the third loops extends about a corresponding third axis that that extends through a space within a radially adjacent one of the second loops.

22. The method of claim 21, wherein the implant is positioned, in the secondary configuration, such that each of the third loops is an open loop that extends partially about the third axis.

23. The method of claim 21, wherein the implant is positioned, in the secondary configuration, such that each of the third loops is a closed loop that extends completely about the third axis.

24. The method of claim 17, wherein the implant is positioned, in the secondary configuration, such that the first consecutive loops form a radially innermost section of the implant.

25. The method of claim 17, wherein the implant is positioned, in the secondary configuration, such that the second consecutive loops form a radially outermost section of the implant.

26. The method of claim 17, wherein the implant is positioned within an aneurysm.

27. An embolic implant comprising:
a strand forming, in a relaxed state, (i) first consecutive loops in a first cycle along a first length of the strand and (ii) second consecutive loops in a second cycle along a second length of the strand;
wherein each of the first loops extends about a corresponding first axis and has a first loop shape;
wherein each of the second loops extends about a corresponding second axis that that extends through a space within a radially adjacent one of the first loops and has a second loop shape, different from the first loop shape.

28. The embolic implant of claim 27, wherein each of the first loops is an open loop that extends partially about the first axis, and each of the second loops is a closed loop that extends completely about the second axis.

29. The embolic implant of claim 27, wherein a minimum cross-sectional dimension of the first loops is greater than a minimum cross-sectional dimension of the second loops.

30. The embolic implant of claim 27, wherein a maximum cross-sectional dimension of the first loops is greater than a maximum dimension of the second loops.

31. The embolic implant of claim 27, wherein each of the first loops forms a polygon.

32. The embolic implant of claim 27, wherein each of the second loops forms a circle or an arc of an incomplete circle.

33. A mandrel for forming an embolic implant, the mandrel comprising:
a core; and
a plurality of posts extending from a surface of the core, each of the posts extending along an axis and having (i) a base section with a first on-face shape and (ii) an extension section with a second on-face shape, different from the first on-face shape.

34. The mandrel of claim 33, wherein the base section is between the core and the extension section.

35. The mandrel of claim 33, wherein a minimum cross-sectional dimension of the base section is greater than a minimum cross-sectional dimension of the extension section.

36. The mandrel of claim 33, wherein a maximum cross-sectional dimension of the base section is greater than a maximum cross-sectional dimension of the extension section.

37. The mandrel of claim 33, wherein the base section forms a polygon in cross-section.

38. The mandrel of claim 33, wherein the extension section forms a circle in cross-section.

39. An embolic implant, comprising:
a strand forming, in a relaxed state, first loops and second loops in one or more cycles along a length of the strand;
wherein the first loops lie in separate planes on opposite sides of a center of the implant, wherein the first loops both extend about a shared first axis;
wherein each of the second loops extends about one of a plurality of second axes that is not parallel with any other of the second axes of any other of the second loops.

40. The embolic implant of claim 39, wherein each of the second axes intersects with the first axis to form a first angle and a second angle, different from the first angle.

41. The embolic implant of claim 39, wherein each of the second loops is closer to one of the first loops than it is to another of the first loops.

42. The embolic implant of claim 39, wherein some of the second loops are closer to a first one of the first loops than to a second one of the first loops and others of the second loops are closer to the second one of the first loops than to the first one of the first loops.

43. A mandrel for forming an embolic implant, the mandrel comprising:
a core;
first posts extending from a surface of the core on opposite sides of the core and along a shared first axis; and
second posts extending from the surface of the core along corresponding second axes, wherein each of the second axes is not parallel with a second axis of any other second post.

44. The mandrel of claim 43, wherein each of the second axes intersects with the first axis to form a first angle and a second angle, different from the first angle.

45. The mandrel of claim 43, wherein each of the second posts is closer to one of the first posts than another of the first posts.

46. The mandrel of claim 43, wherein some of the second posts are closer to a first one of the first posts than to a second one of the first posts and others of the second posts are closer to the second one of the first posts than to the first one of the first posts.

47. An embolic implant comprising:
a strand forming, in a relaxed state, first loops along a first section of the strand and second loops along a second section of the strand;
wherein each of the first loops extends about one of a plurality of first axes;
wherein each of the second loops extends about one of a plurality of second axes;
wherein the first axes intersect at a first region, and the second axes intersect at a second region, distinct from the first region;
wherein a total number of first loops is different from a total number of second loops.

48. The embolic implant of claim 47, wherein the first region is located between at least two first loops, and the second region is located between at least two second loops.

49. The embolic implant of claim 47, wherein the first region is located outside a space bounded by second loops, and the second region is located outside a space bounded by first loops.

50. The embolic implant of claim 47, wherein the maximum dimension of the first section is different from a maximum dimension of the second section.

51. The embolic implant of claim 47, wherein the maximum dimension of the first loops is different from a maximum dimension of the second loops.

52. The embolic implant of claim 47, wherein the first axes are orthogonal to an axis extending through the first region and the second region, and the second axes are transverse to the axis.

53. The embolic implant of claim 47, wherein some of the first loops form, in the relaxed state, a first three-dimensional polyhedral shape, and others of the first loops form, in the relaxed state, a second three-dimensional polyhedral shape, different than the first three-dimensional polyhedral shape.

54. The embolic implant of claim 47, the strand further forming third loops in a third section, wherein each of the third loops and extends about a third axis, wherein the third axes intersect at a third region, distinct from the first region and the second region.

55. A mandrel for forming an embolic implant, the mandrel comprising:
a first core;
first posts extending from a surface of the first core along corresponding first axes, the first axes intersecting at a first region within the first core;
a second core;
second posts extending from a surface of the second core along corresponding second axes, the second axes intersecting at a second region within the second core and distinct from the first region;
wherein a total number of first posts is different from a total number of second posts.

56. The mandrel of claim 55, wherein the first region is located outside the second core, and the second region is located outside the first core.

57. The mandrel of claim 55, wherein the maximum cross-sectional dimension of the first core is different from a maximum cross-sectional dimension of the second core.

58. The mandrel of claim 55, wherein the maximum cross-sectional dimension of the first posts is different from a maximum cross-sectional dimension of the second posts.

59. The mandrel of claim 55, wherein the first axes are orthogonal to an axis extending through the first region and the second region, and the second axes are transverse to the axis.

60. The mandrel of claim 55, further comprising:
a third core; and
third posts extending from a surface of the third core along corresponding third axes, the third axes intersecting at a third region distinct from each of the first region and the second region.
